(19) United States
(12) Patent Application Publication
(10) Pub. No.: US 2026/0311886 A1
(43) Pub. Date: Oct. 8, 2026

(54) EARLY DETECTION OF NEUROCOGNITIVE DECLINE USING MULTI-MODAL MOBILE-DEVICE BASED ASSESSMENT OF PERSONAL PHOTO INTERACTIONS

(71) Applicant: Dabble Health, Inc., Newark, CA (US)

(72) Inventors: Lawrence M. Chernin, Newark, CA (US); Bryan D. Macrie, Hinsdale, IL (US); Min Lang, Cambridge, MA (US); Brooke A. Albright, Temecula, CA (US)

(21) Appl. No.: 19/633,828

(22) Filed: Mar. 30, 2026

Related U.S. Application Data
(60) Provisional application No. 63/782,435, filed on Apr. 2, 2025.

(51) Int. Cl. G16H 50/30; A61B 5/00; A61B 5/11; A61B 5/16; G06T 7/00; G06T 7/20

(52) U.S. Cl. CPC G16H 50/30; A61B 5/1101; A61B 5/163; A61B 5/165; A61B 5/4082; A61B 5/4088; A61B 5/4803; A61B 5/748; G06T 7/0012; G06T 7/20; G06T 2207/30201

(57) ABSTRACT

A method performed by a mobile computing device may comprise accessing, by a mobile computing device, a plurality of personal photographs associated with a user of the mobile computing device. The method further may comprise executing, by the mobile computing device, at least a portion of a multi-activity neurocognitive assessment of a user of the mobile computing device. The multi-activity neurocognitive assessment may include repeatedly collecting multi-modal interaction data by a plurality of sensors of the mobile computing device. The multi-modal interaction data may result from engagement, by the user, with a plurality of personalized neurocognitive activities generated by the mobile computing device using the plurality of personal photographs. The method may further comprise triggering, by the mobile computing device, detection of deviation from a baseline indicative of potential neurocognitive change in the user, the detection being based on evaluation of the collected multi-modal interaction data.

### CROSS-REFERENCE TO RELATED APPLICATIONS

This application claims the benefit of US provisional patent application no. 63/782,435, filed on Apr. 2, 2025 and titled, “Early Detection of Cognitive Decline Using AI-Powered Analysis of Personal Photo Interactions,” which is incorporated herein by reference in its entirety.

### TECHNICAL FIELD

The present disclosure relates to the field of neurocognitive health assessment and monitoring, and more specifically to computer-implemented systems and methods for early detection and continuous monitoring of mild neurocognitive impairment and neurocognitive decline through analysis of user interactions with personalized photo-based activities using artificial intelligence and machine learning technologies.

### BACKGROUND

The cognitive health crisis in America and internationally presents an urgent challenge. At present, about 6.9 million Americans live with Alzheimer's disease and an estimated 17% of adults over 65 experience Mild Cognitive Impairment (MCI). Current research indicates that approximately 47 million older adults may have pre-clinical Alzheimer's Disease, creating a critical need for early detection methods. This widespread impact extends to over 11 million family caregivers, highlighting the societal significance of this healthcare challenge.

Traditional cognitive assessment methodologies include the Mini-Mental State Examination (MMSE) and Montreal Cognitive Assessment (MoCA). These methodologies suffer from several limitations. For example, the tests provide only periodic snapshots rather than continuous monitoring. Additionally, the formal testing environment can affect results by inducing performance anxiety in the evaluation subject. Further, these traditional assessments may include cultural and/or educational bias and may not account for diverse backgrounds. They also may not reflect real-world cognitive function. Furthermore, traditional methods often fail to identify decline during pre-clinical and early MCI stages. Moreover, traditional assessments can be tedious and fail to maintain user participation.

More recently, digital cognitive assessment platforms have emerged as an improvement over traditional pen-and-paper methods. Tools such as Linus Health, Cogstate, BrainCheck, and Neurotrack have made real progress — enabling remote testing, objective scoring, and broader accessibility. But even more modern solutions like these have significant gaps. For example, they rely on generic, standardized stimulus materials and therefore overlook autobiographical memory and social-emotional dimensions. They are also episodic, i.e., used periodically rather than continuously, and they do not account for how each individual naturally varies from day to day. Further, they are generally limited to cognitive domains and do not extend to motor function, movement disorders, or fall risk. Additionally, they are designed primarily for clinical settings rather than for everyday, unsupervised use by the individual to be assessed.

### SUMMARY

The system introduced here includes a neurocognitive impairment detection (NID) system (hereinafter also called “the system introduced here” or simply “the system”) that performs neurocognitive assessment through artificial intelligence (AI) powered analysis of a mobile device user’s interactions with personal digital photographs on the mobile device. The system introduced here uses personal photographs of the user in performing neurocognitive assessment of the user and may be based on multi-modal data acquired by the mobile device during the user’s interactions with games or game-like activities administered to the user by the mobile device, in relation to the personal photographs. The terms “cognitive” and "neurocognitive" are generally used interchangeably in the following description to refer not only to thinking capability, but also to other brain functions, such as perceptual-motor functions, speech/language, and executive functions.

In one general aspect of the system introduced here, a method may include accessing, by a mobile computing device, a plurality of personal photographs associated with an user of the mobile computing device. The method may also include executing, by the mobile computing device, at least a portion of a multi-activity neurocognitive assessment of an user of the mobile computing device, where the multi-activity neurocognitive assessment includes repeatedly collecting multi-modal interaction data by a plurality of sensors of the mobile computing device, the multi-modal interaction data resulting from engagement, by the user of the mobile computing device, with a plurality of personalized neurocognitive activities generated by the mobile computing device using the plurality of personal photographs associated with the user of the mobile computing device. The method may furthermore include triggering, by the mobile computing device, detection of deviation from a baseline indicative of potential neurocognitive change in the user, the detection being based on evaluation of the collected multi-modal interaction data. The triggering may include invoking an evaluation process within the mobile device, or causing transmission, by the mobile computing device, of the collected multi-modal interaction data to a server system configured to evaluate the multi-modal interaction data to detect deviation from a baseline indicative of potential neurocognitive change. Other implementations of this aspect include one or more corresponding computer systems, apparatus, and computer programs recorded on one or more computer storage devices, each configured to perform the actions of the methods.

Implementations of the method may include one or more of the following features. The multi-modal data may include: touchscreen input data acquired from a touchscreen of the mobile computing device; and speech data acquired from a microphone of the mobile computing device. The multi-modal data further may include eye motion data and/or facial expression data acquired by a camera of the mobile computing device. The multi-modal data further may include data indicative of hand tremor frequency and amplitude acquired by an accelerometer of the mobile computing device and/or other data indicative of a movement disorder. The multi-activity neurocognitive assessment may include administering a multi-activity challenge involving a plurality of different activities, to the user. Implementations of the described system may include hardware, a method or process, or a computer tangible medium.

In another general aspect of the system introduced here, a mobile computing device may include a touchscreen, a microphone, a wireless transceiver configured to enable communications over a wireless communication network, and at least one processor. The mobile computing device may further include at least one memory storing instructions, execution of which by the at least one processor causes the mobile computing device to perform a process. The process may include accessing a plurality of personal photographs associated with an user of the mobile computing device; executing at least a portion of a multi-activity neurocognitive assessment of an user of the mobile computing device, where the multi-activity neurocognitive assessment includes repeatedly collecting multi-modal interaction data by a plurality of sensors of the mobile computing device, including the touchscreen in the microphone, the multi-modal interaction data resulting from engagement, by the user of the mobile computing device, with a plurality of personalized neurocognitive activities generated by the mobile computing device using the plurality of personal photographs associated with the user of the mobile computing device; and triggering detection of deviation from a baseline indicative of potential neurocognitive change in the user, the detection being based on evaluation of the collected multi-modal interaction data. The triggering may include invoking an evaluation process within the mobile device, or causing transmission, by the mobile computing device, of the collected multi-modal interaction data to a server system configured to evaluate the multi-modal interaction data to detect deviation from a baseline indicative of potential neurocognitive change. Other implementations of this aspect include corresponding computer systems, apparatus, and computer programs recorded on one or more computer storage devices, each configured to perform the actions of the methods.

Implementations of the method introduced may include one or more of the following features. The multi-modal data further may include at least one of: eye motion data and/or facial expression data acquired by a camera of the mobile computing device; or data indicative of hand tremor frequency and amplitude acquired by an accelerometer of the mobile computing device and/or other data indicative of a movement disorder. The multi-activity neurocognitive assessment may include administering a multi-activity challenge involving a plurality of different activities, to the user. The multi-activity challenge protocol may include: a visual pattern matching activity; a jigsaw puzzle activity; and a memory story narration activity requiring a spoken response by the user. Implementations of the described system may include hardware, a method or process, or a computer tangible medium.

In yet another general aspect of the system introduced here, a method may include receiving, by a server computer system, multi-modal interaction data collected by a plurality of sensors of a mobile computing device that is separate from the server computer system, the multi-modal interaction data resulting from engagement, by an user of the mobile computing device, with a plurality of personalized neurocognitive activities generated by the mobile computing device using personal photographs associated with the user. The method may also include processing, by the server computer system, the multi-modal data by using one or more machine learning models to detect deviation from a baseline indicative of potential neurocognitive change. The method may furthermore include outputting, by the server computer system, a result of the processing to a provider interface. Other implementations of this aspect include corresponding computer systems, apparatus, and computer programs recorded on one or more computer storage devices, each configured to perform the actions of the methods.

Implementations of the method may include one or more of the following features. The provider interface may include a healthcare provider interface configured to enable controlled access to a result of the processing by a healthcare provider. The result may include a difficulty-normalized composite neurocognitive health score and a plurality of domain scores related to the user of the mobile computing device, the plurality of domain scores corresponding to a plurality of neurocognitive domains. The processing may include evaluating temporal response patterns and generating accuracy metrics related to the engagement by the user with the personalized neurocognitive activities generated by the mobile computing device. The personalized neurocognitive activities generated by the mobile computing device may include a multi-activity challenge protocol. The multi-activity challenge protocol may include: a visual pattern matching activity; a jigsaw puzzle activity; and a memory story narration activity requiring a spoken response by the user. The method may include: qualifying, by the server system, each photograph of the plurality of personal photographs for use in one or more of the plurality of personalized neurocognitive activities, by estimating a depth of subject matter depicted in the photograph. The method may include: maintaining, by the server system, a longitudinal per-user performance baseline for the user; and generating, by the server system, the result of the processing by evaluating the performance of the user in the personalized neurocognitive activities against the per-user performance baseline for the user. The multi-modal data may include: touchscreen input data acquired from a touchscreen of the mobile computing device; and speech data acquired from a microphone of the mobile computing device. The multi-modal data further may include eye motion data and/or facial expression data acquired by a camera of the mobile computing device. The multi-modal data further may include data indicative of hand tremor frequency and amplitude acquired by an accelerometer of the mobile computing device and/or other data indicative of a movement disorder. Implementations of the described system may include hardware, a method or process, or a computer tangible medium.

In still another general aspect of the system introduced here, a server system may include at least one processor, and at least one machine-readable program medium storing instructions, execution of which by at least one processor causes the server system to perform a process. The process may include receiving multi-modal interaction data collected by a plurality of sensors of a mobile computing device that is separate from the server system, the multi-modal interaction data resulting from engagement, by an user of the mobile computing device, with a plurality of personalized neurocognitive activities generated by the mobile computing device using personal photographs associated with the user; processing the multi-modal data by using one or more machine learning models to detect deviation from a baseline indicative of potential neurocognitive change; and outputting a result of the processing to an user interface. Other implementations of this aspect include corresponding computer systems, apparatus, and computer programs recorded on one or more computer storage devices, each configured to perform the actions of the methods.

Implementations of the server system may include one or more of the following features. The result may include a difficulty-normalized composite neurocognitive health score and a plurality of domain scores related to the user of the mobile computing device, the plurality of domain scores corresponding to a plurality of neurocognitive domains. The processing may include evaluating temporal response patterns and generating accuracy metrics related to the engagement by the user with the personalized neurocognitive activities generated by the mobile computing device. The personalized neurocognitive activities generated by the mobile computing device include a multi-activity challenge protocol, including: a visual pattern matching activity; a jigsaw puzzle activity; and a memory story narration activity requiring a spoken response by the user. The processing may include: qualifying each photograph of the plurality of personal photographs for use in one or more of the plurality of personalized neurocognitive activities, by estimating a depth of subject matter depicted in the photograph. The multi-modal data may include: touchscreen input data acquired from a touchscreen of the mobile computing device; and speech data acquired from a microphone of the mobile computing device. The multi-modal data further may include at least one of: eye motion data and/or facial expression data acquired by a camera of the mobile computing device; or data indicative of hand tremor frequency and amplitude acquired by an accelerometer of the mobile computing device and/or other data indicative of a movement disorder. Implementations of the described system may include hardware, a method or process, or a computer tangible medium.

### BRIEF DESCRIPTION OF THE DRAWINGS

FIG. 1A shows an example of a network environment in which the system introduced here can be implemented.

FIG. 1B shows an example of the system architecture of the system introduced here.

FIG. 2 is a data flow diagram illustrating the movement of information in the system from user interaction to clinical insight generation.

FIG. 3A shows an example of the processing flow of the photo-to-activity conversion process of the system.

FIG. 3B shows an example of the processing flow of the metadata analysis stage of the photo-to-activity conversion process of the system.

FIG. 3C shows an example of the processing flow of the visual content analysis stage of the photo-to-activity conversion process of the system.

FIG. 3D shows an example of the processing flow of the activity generation and psychometric validation stage of the photo-to-activity conversion process of the system.

FIG. 4 shows an example of the processing flow of the neurocognitive decline detection process of the system.

FIG. 5A illustrates a first portion of an example of the main functional components of the AI engine.

FIG. 5B illustrates a second portion of the example of the main functional components of the AI engine, corresponding to the first portion in FIG. 5A.

FIG. 6 illustrates an example of a dashboard display that may be generated by the clinical dashboard interface.

FIG. 7 illustrates an example of the functionality of a biometric processing component of the system.

FIG. 8 shows an example of a process that may be performed by the anxiety detection engine.

FIG. 9 illustrates an example of an enterprise interface dashboard that may be generated by the system.

FIG. 10 depicts an example of a display screen of the multi-site management user interface that may be generated by the system.

FIG. 11 is a flow diagram illustrating an example of a process that may be performed by a mobile device as part of a neurocognitive assessment of a user.

FIG. 12 is a flow diagram illustrating an example of a process that may be performed by a server as part of a neurocognitive assessment of a user.

FIG. 13 is a block diagram of a computer system that may be used to implement some or all of the techniques introduced above, or any portion(s) thereof.

### DETAILED DESCRIPTION

Note that references in this description to certain features or functions being “optional,” “optionally present” or similar phrasing are intended only for emphasis and do not imply that other features that are not so characterized are necessary or mandatory.

### I. Introduction

The neurocognitive assessment literature distinguishes between performance on a task and the rate of learning across repeated exposures to the same task. In healthy brains, repeated exposure to personally meaningful stimuli produces measurable improvement curves as neural circuits strengthen through use — a direct expression of neuroplasticity. In pre-clinical and early-stage neurocognitive decline, this neuroplastic strengthening is impaired: the learning curve across repeated exposures is shallower than in age-matched healthy controls, even before overall performance degrades to levels that would trigger failure on standard instruments such as the MoCA. This deviation in the learning curve trajectory — rather than any single performance measurement — can represent a pre-clinical signal that the system introduced here detects.

The system introduced here includes a neurocognitive impairment detection (NID) system (hereinafter also called “the system introduced here,” “the NID system” or simply “the system”). The NID system uses personal photographs of a person to be evaluated (the “test subject”) in performing neurocognitive assessment. At least a portion of the system (and in some implementations, all of the system) may be implemented in a mobile device (e.g., a smartphone, tablet or laptop computer) used by the test subject. As part of the neurocognitive assessment, the mobile device may acquire and analyze multi-modal data indicative of a user’s interactions with the mobile device during activities related to the user’s personal photographs and administered to the user by the mobile device.

The use of personal photographs relates essentially to signal-to-noise ratio: photographs from a user’s own life — whether of people, places, events, or objects — tend to produce strong, consistent, high-amplitude learning curves in healthy individuals, because the content is inherently engaging and personally salient. Generic, stock, or standardized photographs tend to produce weak and inconsistent learning curves with high inter-individual variance. Against the strong, consistent baseline that personal photographs can establish, the shallowing of the learning curve in early neurocognitive decline is a detectable and statistically reliable signal. Against the weak, noisy baseline that generic photographs provide, the same degree of neuroplastic degradation is masked by normal variability. The use of personal photographs is therefore a significant advance in achieving pre-clinical detection sensitivity.

A further advantage of the system over standardized neurocognitive assessments such as the MoCA is the lack of susceptibility to a user’s improvement in performance due to mere practice (“practice effects”). The MoCA and similar instruments are administered using fixed, standardized stimuli. Consequently, repeated exposure to the same tasks allows users to improve their scores through familiarity rather than genuine neurocognitive improvement, inflating scores in both healthy and mildly impaired populations and reducing the instrument’s sensitivity to true change over time. The system introduced here avoids this limitation through three mechanisms: (i) photographs are selected from the user’s own library with freshness weighting that deprioritizes recently shown images, so that stimuli remain novel across sessions; (ii) difficulty is automatically adjusted to maintain consistent challenge level regardless of learning; and (iii) the longitudinal baseline tracks intra-individual change rather than absolute performance against fixed task norms. The system therefore measures genuine neurocognitive trajectory rather than task familiarity, and is not confounded by the practice effects that limit the longitudinal utility of standardized assessments.

The MoCA measures snapshot performance on standardized abstract tasks. In contrast, the system introduced here measures neuroplastic learning curve dynamics and multi-modal behavioral biomarkers collected during naturalistic, personally meaningful activities. These are complementary constructs. At least one study has demonstrated correlation between outputs of the system introduced here and MoCA scores alongside other clinical indicators of neurocognitive impairment, establishing clinical alignment rather than equivalence. Longitudinal scoring methodology and expanded clinical validation are the subject of separate patent applications.

Despite extensive research identifying affected neurocognitive domains, the field lacks effective methods to assess neuroplasticity – the brain's ability to form new connections in response to learning and experiences. As established in recent research, neuroplasticity is fundamentally linked to neurocognitive health, particularly in aging populations. Studies show that age-related neurocognitive decline is associated with alterations in synaptic plasticity, which is critical for learning and memory (see, e.g., Navakkode, S., et al, “Neural Ageing and Synaptic Plasticity: Prioritizing Brain Health in Healthy Longevity,” Frontiers in Aging Neuroscience, 16, 1428244 (2024)).

In people with Alzheimer's disease, neuroplasticity becomes impaired, making it harder to learn new information or recall memories. A key indicator of this impairment is reaction time to visual stimuli, particularly when viewing the same images repeatedly. Research has demonstrated that measuring reaction times to repeated photographs offers valuable insights into neuroplastic processes. In healthy brains, reaction times improve with repeated exposure as neural pathways strengthen – a direct manifestation of neuroplasticity. However, in individuals with neurocognitive decline, these reaction time improvements are diminished or absent, reflecting compromised neuroplastic function.

Personal photographs represent a unique opportunity to assess these neuroplasticity mechanisms, as they contain emotional significance and contextual elements that activate multiple neural pathways simultaneously. Current assessment methods fail to leverage these personally meaningful stimuli to measure reaction time patterns that reveal underlying neuroplastic processes, particularly in early disease stages when intervention would be most effective.

Despite extensive research identifying affected neurocognitive domains, the field lacks validated digital biomarkers for continuous monitoring through natural interactions. It also lacks methodologies for personalizing assessments based on individual life experiences. Further, the field lacks systems for early detection of subtle neurocognitive changes. Additionally, the field lacks engaging assessment tools that encourage regular use.

Prior attempts to address these limitations have included: digital versions of traditional neurocognitive tests; game-based neurocognitive assessments; passive monitoring systems; and standardized photo-based memory tests. However, these solutions have not successfully combined personalization, engagement, and clinical validity in a way that enables early detection of neurocognitive decline.

### II. System Overview

The NID system introduced here provides a solution for neurocognitive assessment through artificial intelligence (AI) powered analysis of a mobile device user’s interactions with personal digital photographs on the mobile device. The system transforms the photographs into personalized neurocognitive activities and can include advanced machine learning algorithms to analyze behavioral patterns during user engagement.

Implementations of the system introduced here can include some or all of the following features:
1. Conversion of personal photos into validated neurocognitive assessment activities
2. Multi-modal data analysis of user interactions, including temporal, spatial, and semantic patterns
3. Depth-based photo authenticity verification
4. Adaptive difficulty adjustment based on real-time performance metrics
5. Multi-game daily challenge and multiple performance metrics
6. Per-user longitudinal baseline and on-device/cloud implementations
7. Integration of social engagement features to maintain long-term participation.
8. Neuroplasticity-targeted assessment
9. Integration with biometric data from wearable devices.
10. Enterprise-level analytics for senior care, assisted living, and caregiver organizations

### A. Conversion of Personal Photos into Validated Neurocognitive Assessment Activities

The system uses photographs drawn from the user’s own camera roll library — which may include any personally meaningful content including people, locations, events, and objects — as neurocognitive assessment stimuli. Personal photographs produce stronger and more consistent neuroplastic learning curves in healthy brains than generic stimuli, enabling reliable detection of the learning curve shallowing that characterizes pre-clinical neurocognitive decline. In certain implementations, the system analyzes personal photographs using computer vision, for example, to identify people, places, objects, and relationships within images. It then creates standardized neurocognitive tasks that leverage these personalized elements while maintaining scientific validity. This can be achieved through calibrated difficulty levels, normalized scoring methods, established correlations with clinical measures such as MMSE, and controls for variables such as image quality and emotional content that might otherwise affect assessment accuracy. The photo selection algorithm optimizes for content variety, session-to-session freshness, and interestingness score to maintain engagement and ecological validity across longitudinal assessments.

### B. Multi-Modal Data Analysis of User Interactions, Including Temporal, Spatial, and Semantic Patterns

In certain implementations, the platform captures detailed interaction data including response times, touch precision, gesture patterns, navigation efficiency, and content engagement metrics, and touchscreen tap, drag and pressure for movement disorder tracking. Machine learning models analyze these multi-dimensional behavioral patterns to identify subtle indicators of neurocognitive function that might not be apparent through traditional assessment methods.

In certain implementations, the system simultaneously captures and fuses interaction data from at least two hardware sensors of a mobile computing device. For example, a touchscreen sensor captures the user’s tap events, drag gesture trajectories, touch pressure force, and response timing to millisecond precision; and concurrently, and for example, the microphone captures the user’s speech during voice-prompted memory story narration; and/or an accelerometer in the mobile device captures the user’s hand tremor frequency, amplitude, and micro-movement patterns as motor biomarkers, concurrently with the other mentioned sensor data capture. Additionally or alternatively, the front-facing camera of the mobile device may capture saccadic eye movement patterns and facial expressions of the user, concurrently with the other mentioned sensor data capture.

### C. Depth-Based Photo Authenticity Verification

To ensure neurocognitive assessment activities are based on genuine personal memories rather than reproduced or synthetic images, the system may apply a depth estimation/verification process to each photograph prior to its use as a neurocognitive stimulus. In certain implementations, the depth estimation process first checks for embedded depth data from smartphone Portrait mode capture or LiDAR sensor data where available. Where embedded depth data is absent, the system may apply a machine learning depth estimation model to generate a depth map. The system then may analyze depth variance across the image: genuine real-world photographs of scenes and subjects exhibit substantial depth variation, while screenshots, photographs of printed photographs, and downloaded images (including memes and reproduced content) exhibit characteristically low depth variance consistent with a flat plane. Photographs that do not pass depth verification can be excluded from use as neurocognitive assessment stimuli. This verification serves the additional function of gating display of GPS location metadata: location information is displayed only for photographs that pass depth verification, preventing misleading location data carried by photographs of printed pictures.

### D. Adaptive Difficulty Adjustment Based on Real-Time Performance Metrics

In certain implementations, by using reinforcement learning techniques, the system continuously monitors performance and dynamically adjusts task difficulty to maintain optimal challenge levels. This personalized approach prevents ceiling and floor effects while keeping users engaged, resulting in more sensitive measurement across the neurocognitive spectrum.

The automatic difficulty adjustment can be based on real-time performance signals including accuracy rate, response latency, error frequency, and task completion rate, targeting an optimal challenge level for each user. Difficulty parameters are also user-configurable, allowing the user or caregiver to set difficulty ceilings, floors, or preset profiles per game type. All neurocognitive scores can be normalized for the active difficulty level, enabling valid longitudinal comparison across sessions completed at different difficulty settings. Adaptive difficulty scaling can be implemented across two or more game-based assessment modalities, examples of which are discussed below.

### E. Multi-Game Daily Challenge and Multiple Performance Metrics

The system in some implementations may administer a structured daily challenge comprising multiple sequential games, such as:
(i) a personal photo matching game where the system captures and analyzes, for example, reaction time, accuracy, signal detection d-prime, and composite game scores;
(ii) a personal photo based jigsaw puzzle game where the system captures and analyzes, for example, solve times and completion rates; and
(iii) a memory story narration task in which the user provides spoken narration about their personal photos, where the system captures and analyzes, for example, speech fluency parameters, voice acoustic biomarkers, speech production analysis, accuracy of the user’s characterization of photo content, neurocognitive domain scores, word trace assessment (e.g., a post-story game), and accelerometer-based tremor analysis (during recording).

The term “game” is used herein to mean any goal-oriented activity designed to provide at least some entertainment value for the user. The system measures multiple performance metrics of the user in playing the games (e.g., 12 metrics in some implementations), and those metrics then constitute a core feature set for machine learning classification.

### F. Per-User Longitudinal Baseline and On-Device/Cloud Implementations

The system may establish an individualized performance baseline from each user’s own prior session data, enabling detection of intra-individual neurocognitive change before any population-level threshold is crossed. In at least one on-device implementation, all processing is performed locally on the mobile computing device and no personal photographs or derived health data are transmitted to any remote server. In this description, the term “on-device” refers to features and/or functionality being implemented on a mobile device. In alternative implementations, at least some of the disclosed processing may be performed on one or more remote servers.

The system in at least some implementations specifically measures how reaction times and recognition accuracy change when users encounter the same photos multiple times across sessions. These changes in response patterns can provide unique insights into neuroplastic function—the brain's ability to modify neural connections—which is a critical marker for neurocognitive health that traditional assessments typically fail to capture.

### G. Integration of Social Engagement Features to Maintain Long-Term Participation

In certain implementations, the platform incorporates photo sharing, collaborative reminiscence activities, and family participation options that transform neurocognitive assessment into a meaningful social experience. These features significantly improve long-term engagement rates by embedding assessment within natural social interactions that users find inherently rewarding.

### H. Neuroplasticity-Targeted Assessment

The system measures the brain's ability to form and strengthen neural connections through analysis of reaction times and learning patterns during repeated exposure to personalized photographic stimuli.

### I. Integration with Biometric Data from Wearable Devices

The system can include integration with biometric data from wearable devices (e.g., smartwatches) or health platforms to account for physiological factors affecting test performance: The platform in some implementations can connect with wearable devices directly or access data from health platforms such as Apple HealthKit to incorporate physiological metrics including heart rate, HRV, and skin temperature. This capability provides valuable context for interpreting neurocognitive performance by accounting for test anxiety, sleep quality, and other physiological factors that can impact assessment results.

### J. Enterprise-Level Analytics

For organizational implementations, the system provides population-level analytics, resource optimization tools, and quality metrics reporting. These enterprise capabilities enable senior care facilities to identify residents at highest risk, allocate intervention resources effectively, coordinate care across staff members, and document neurocognitive health outcomes for quality reporting requirements.

By incorporating some or all of the above mentioned features, the system introduced here provides several advantages over known prior techniques, including:
1) Early detection of subtle neurocognitive changes through continuous monitoring
2) Reduced performance anxiety through naturalistic assessment
3) Increased ecological validity through use of personally meaningful content
4) Enhanced engagement through social features and photo enhancement tools
5) Improved clinical utility through integration with existing healthcare systems
6) Optional physiological context through biometric integration
7) Enterprise-level insights for care organizations

### III. Overall Architecture and Functionality

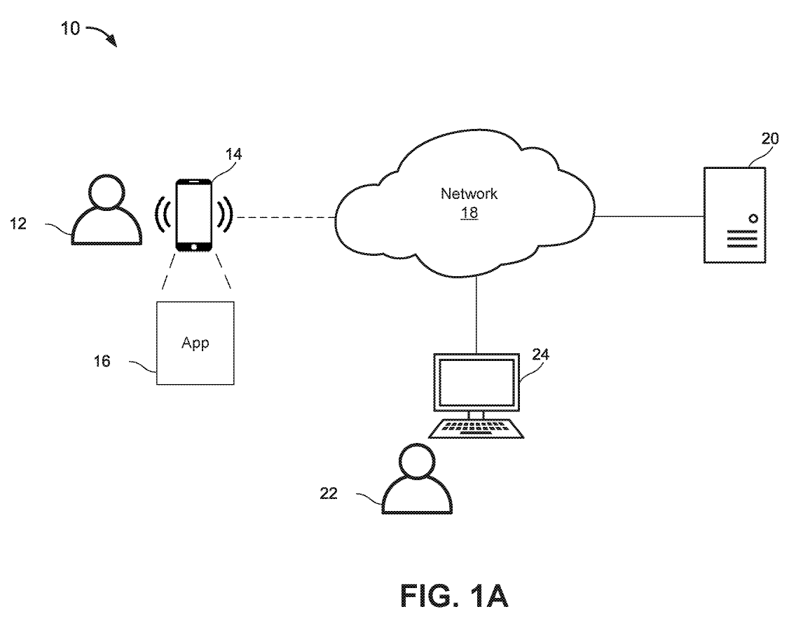

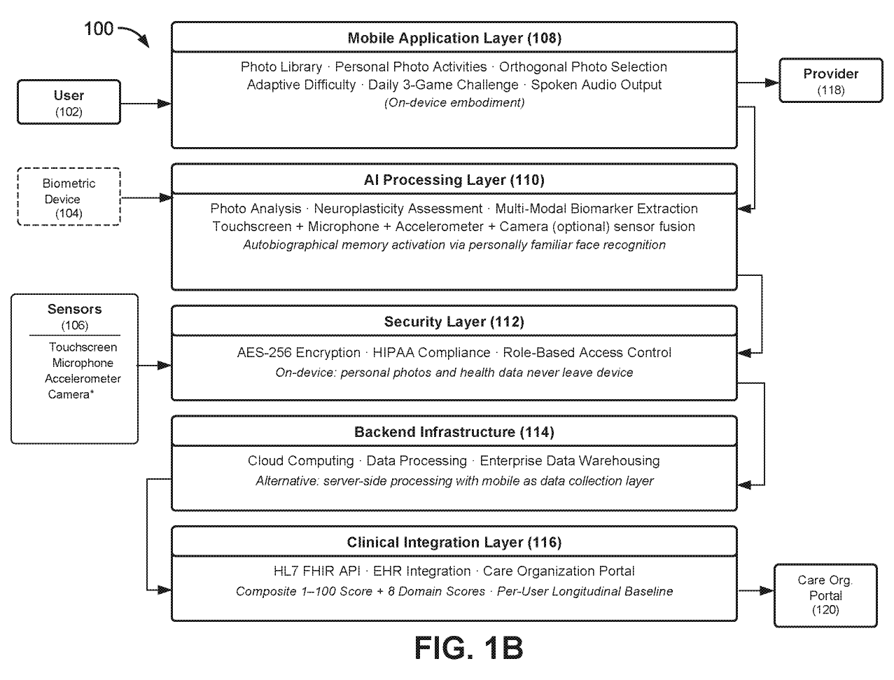

FIG. 1A illustrates an example of a network environment 10 in which the NID system introduced here can be implemented. The network environment 10 includes a mobile computing device 14, such as a smartphone, tablet, or other portable electronic device, which is utilized by a user 12. In this context, user 12 represents a test subject to be evaluated for potential MCI or other forms of neurocognitive decline. A mobile application (“app”) 16 runs on the mobile computing device 14 to administer personalized neurocognitive assessment activities. At least some of the functionality of the NID system may be implemented in the mobile app 16, as discussed further below.

The mobile computing device 14 is communicatively coupled to a network 18, which may comprise a wireless communication network (e.g., a Wi-Fi network, cellular network), the Internet, or a combination thereof. Also connected to the network 18 is a server system 20. In at least some embodiments, the server system 20 may implement some or all of the processing functions of the NID system as an alternative or supplement to the processing performed by the mobile application 16, as describe further below. A second user 22, representing a user associated with a healthcare provider, assisted living facility, or similar care-giving entity, may interface with the system via a provider computing device 24 connected to the network 18. This allows user 22 to access a web-based portal to monitor the neurocognitive health and assessment results of user 12.

FIG. 1B shows an example of the system architecture of the system introduced here. In some implementations, the system 100 comprises four primary software components, or layers: a mobile application layer (or simply “mobile application”) 108, an AI processing layer 110, a backend infrastructure layer 114, and a clinical integration layer 116. The mobile application layer 108 may represent mobile app 16 in FIG. 1A.

The system 100 may be implemented in an on-device implementation, a server-based implementation, or a hybrid implementation (i.e., combination of on-device and server-based). In an on-device implementation, all processing may be performed locally on the mobile computing device (e.g., mobile device 14 in FIG. 1A) such that no personal photographs or derived health data are transmitted to any remote server. In alternative implementations, some or all processing may be performed on remote servers or cloud computing systems.

### A. Mobile Application Layer

The mobile application layer 108 serves as the user interface and data collection point. It handles photo management through a custom image processing process that optimizes photos for neurocognitive assessment while preserving emotional and contextual elements. The mobile application layer 108 can implement a specialized event capture system that hooks directly into the gesture recognition system to record precise timing of interactions with millisecond accuracy. Touch events can be processed through a custom input handling layer that captures not just basic tap data but also pressure variations, micro-movements, and hesitation patterns. The UI can adapt dynamically based on detected user capabilities, automatically adjusting element sizes, spacing, and timing parameters without requiring explicit accessibility settings.

The mobile application layer 108 may include, for example:
1) a custom timing-sensitive interaction capture system capable of detecting millisecond-level hesitations and response pattern changes of a user 102, indicative of early neurocognitive decline, with specialized algorithms for distinguishing between motor and neurocognitive processing delays;
2) a proprietary photo content organization algorithm that analyzes metadata, visual features, and user input to arrange personal images into neurocognitively meaningful sequences optimized for assessment across multiple neurocognitive domains;
3) an adaptive neurocognitive task generation engine with multiple (e.g., 14) distinct task templates that dynamically adjust based on photo content, ranging from simple recognition to complex temporal sequencing and contextual memory challenges;
4) a touch pattern analysis system that captures multiple (e.g., six) dimensions of fine motor control variations, including touch pattern precision, pressure, trajectory, tremor, hesitation, and correction patterns, associated with specific neurocognitive status indicators;
5) a memory-optimized content delivery system implementing a proprietary spaced repetition algorithm with dynamically adjusted intervals to precisely measure memory consolidation and recall patterns; and
6) an emotional content calibration system that analyzes facial expressions, scene composition, and user annotations to balance emotional salience across assessment tasks, thereby preventing emotional bias in neurocognitive measurements.

### B. AI Processing Layer

The AI processing layer 110 may include:
1) a specialized neural network architecture with dedicated pathways for facial recognition memory, location memory, temporal sequencing, and emotional processing, designed specifically for autobiographical memory assessment using personal photos;
2) a multi-domain neurocognitive analysis engine that extracts distinct markers across episodic memory, executive function, attention, and processing speed domains from interaction patterns during photo-based activities;
3) a reaction-time decay analysis system employing proprietary statistical models to measure distinct aspects of neuroplasticity through changes in response patterns to repeated stimuli across varying time intervals; and
4) a neurocognitive-load estimation algorithm that continuously monitors performance indicators to adaptively adjust task difficulty in real time while maintaining assessment validity.

The AI processing layer 110 may employ an AI engine that extracts and analyzes one or more digital biomarkers across several categories. For example, temporal patterns may include response latency distributions, task completion time trajectories, interaction rhythm analysis, time-based error patterns, and activity session duration. Spatial patterns may include touch precision metrics, gesture complexity analysis, navigation efficiency, and screen area coverage. Neurocognitive markers may include memory recall accuracy, sequential ordering performance, and narrative complexity metrics.

Neuroplasticity markers may include eye tracking neurocognitive biomarkers. On mobile devices with a TrueDepth front-facing camera, the system may employ, for example, ARKit ARFaceTrackingConfiguration to capture gaze direction, saccadic eye movements, and pupil dilation at camera frame rate without additional hardware. The following game-specific neurocognitive biomarkers can be acquired:
a. During the memory story narration task (“game”): The system can track gaze patterns while the user views a personal photograph and provides spoken narration. Reduced fixation on faces present in the photograph — particularly faces of personally familiar individuals — is an early marker of prosopagnosia or attentional decline. Saccade latency, defined as time between stimulus onset and initial gaze shift to a new image region, correlates with processing speed and is measured continuously across sessions. Erratic or wandering gaze during narration, quantified as reduced fixation duration and increased saccade frequency relative to the user’s own baseline, is indicative of difficulty with episodic memory retrieval. The system may additionally compute a dual-task attentional cost: if gaze coherence decreases when the user is simultaneously viewing and narrating compared to passive viewing, the magnitude of this difference quantifies attentional reserve — one of the earliest neurocognitive capacities to decline in neurodegenerative disease.
b. During the memory match game: The system may apply pupillometry, measuring pupil dilation as a proxy for neurocognitive load, since pupil diameter increases monotonically with mental effort under stable lighting conditions. The system additionally may track whether the user visually revisits previously revealed card positions between exposures. The ratio of purposeful spatial revisits to random scanning provides a continuous measure of visuospatial working memory strategy quality.
c. During the jigsaw puzzle game: Gaze heat maps reveal problem-solving strategy. A systematic edge-first approach, in which gaze concentrates on boundary pieces before interior pieces, reflects intact executive planning. A scattered trial-and-error approach reflects reduced executive function. Time-to-first-fixation on the correct target placement region measures visual pattern recognition speed. Longitudinal shift from systematic to scattered strategy is a sensitive indicator of executive function decline detectable before accuracy metrics degrade.

All eye tracking metrics can be tracked longitudinally against the per-user baseline. A single session’s saccade latency or fixation coherence score has limited standalone diagnostic value; whereas, a sustained increase over multiple sessions, normalized against the user’s own trajectory, constitutes a sensitive marker of processing speed or attentional decline. This directly extends the system’s existing per-user longitudinal baseline infrastructure to the eye tracking modality.

Photo authenticity via depth estimation: The system can employ a depth estimation process to verify photographs represent genuine camera-captured three-dimensional scenes. The process may first check for hardware-embedded depth data from Portrait mode processing or LiDAR sensor capture. For photographs lacking hardware depth data, an on-device ML depth estimation model (e.g., Depth Anything V2, compiled to the device neural engine via CoreML) can generate a scene depth map from the RGB image. Spatial variance of the depth map can classify authenticity: genuine three-dimensional photographs produce high depth variance; screenshots, photographs of flat printed images, and digital graphics produce low depth variance. Only photographs exceeding the depth variance threshold are used as neurocognitive stimuli. GPS location metadata is displayed only for photographs passing depth verification, preventing misleading location display from photographs of printed pictures carrying incorrect GPS metadata.

### C. Backend Infrastructure

The backend infrastructure 114 may include:
1) a neurocognitive assessment data model with hierarchical organization specifically designed for efficient storage and retrieval of photo-based interaction patterns across multiple time scales (within-session, daily, weekly, monthly, and yearly); and
2) a contextual analysis system that incorporates environmental factors, time-of-day effects, and physiological state data when interpreting neurocognitive performance variations

### D. Clinical Integration Layer

The clinical integration layer 116 may include:
1) a neurocognitive domain mapping visualization with multidimensional representation of six primary neurocognitive domains (memory, attention, executive function, language, visuospatial skills, and processing speed) optimized for clinical interpretation;
2) enterprise analytics system with population segmentation, risk stratification, and intervention prioritization tools designed specifically for senior care environments to optimize resource allocation;
3) longitudinal neurocognitive change detection system using proprietary trend analysis to distinguish between random variability and clinically significant changes with configurable alert thresholds based on rate and pattern of change;
4) care team coordination module with role-specific interfaces tailored to the information needs of different care providers 118 (e.g., physicians, nurses, therapists, and caregivers) with appropriate detail levels, and to generate a portal 120 for access of such information; and
5) quality metrics generation system that automatically documents neurocognitive health interventions and outcomes for regulatory reporting in senior care settings.

### E. Optional Biometric Integration

The system optionally may provide integration with biometric data sources, such as biometric device 104 in FIG. 1B, to enhance assessment accuracy by accounting for physiological factors that can impact neurocognitive performance. This integration can occur through either of two primary methods. Under a direct smartwatch integration method, the system can collect data in real time during assessment sessions, continuously monitor via compatible smartwatch devices, synchronize with the mobile application via Bluetooth, and support low-latency processing for immediate assessment adaptation. Under a health platform integration method, the system can connect to, for example, Apple HealthKit, Google Fit, or other health data platforms, retrieve historical and current biometric data, batch-process physiological trends, and provide permission-based secure access to health records.

1. Biometric Correlation with Neurocognitive Performance

When biometric data is available, the system may analyze physiological indicators to detect and account for test anxiety and other factors that can influence neurocognitive performance. Research has established clear correlations between specific biometric patterns and both neurocognitive function and test anxiety. In particular, elevated heart rate combined with reduced HRV strongly correlates with increased test anxiety, and that anxiety state is associated with diminished neurocognitive performance, particularly in executive function and working memory. The system can analyze real-time heart-rate patterns to identify potential anxiety states that may affect test validity. Decreased peripheral skin temperature is associated with sympathetic nervous-system activation during anxiety states; therefore, the system can monitor temperature trends during assessment sessions to identify stress responses, with sudden temperature drops flagged as potential indicators of anxiety that may impair neurocognitive performance. The system may also incorporate prior sleep data when available from health platforms, because higher HRV during sleep and consistent sleep patterns correlate with improved neurocognitive performance, and this data provides contextual information for interpreting test results.

2. Anxiety Detection and Compensation Methodology

When biometric data is available, the system can employ a multi-step approach to account for the effects of test anxiety on neurocognitive assessment. First, in a pre-test baseline-establishment phase, the system collects resting heart rate, HRV, and skin temperature prior to neurocognitive tasks and establishes individual baseline patterns for comparison during testing. Next, during real-time anxiety monitoring, the system continuously monitors biometric indicators throughout the assessment and applies proprietary algorithms to detect patterns indicative of test anxiety. Then, in an adaptive testing protocol, when significant anxiety indicators are detected, the system may introduce brief relaxation intervals between test components, adjust difficulty progression to reduce anxiety, and flag potentially affected results for contextual interpretation. Finally, in a post-test analysis phase, the system correlates performance metrics with biometric data, identifies performance patterns likely influenced by anxiety versus true neurocognitive status, and applies compensation factors to create anxiety-adjusted scores when appropriate.

3. Clinical Validity Enhancement

Biometric integration can significantly enhance the clinical validity of neurocognitive assessments by reducing false positives through identifying instances in which poor performance may be attributed to anxiety rather than neurocognitive impairment, thereby preventing misclassification of anxious individuals as neurocognitively impaired. Further it can improve test-retest reliability by accounting for day-to-day variations in anxiety levels and providing more consistent assessment results over time. Additionally, biometric integration can increase personalized assessment context by incorporating individual physiological response patterns and recognizing that different individuals exhibit varying biometric responses to similar anxiety levels. Further, it can create holistic assessment parameters that move beyond pure task performance to incorporate physiological state and reflect the interdependent nature of emotional state and neurocognitive function.

### F. Personalization System

Use of reinforcement learning of the AI engine can optimize the user experience through content selection, difficulty adjustment, feature adaptation, and engagement optimization. With respect to content selection, the system can perform photo relevance scoring, emotional significance analysis, memory association mapping, temporal context matching, and evaluation of social connection strength. With respect to difficulty adjustment, the system can apply performance-based scaling, neurocognitive-load balancing, error-rate optimization, time-pressure modulation, and complexity progression. With respect to feature adaptation, the system can simplify the interface, optimize navigation, activate help systems, tune notifications, and adjust social features. With respect to engagement optimization, the system can schedule rewards, balance challenge levels, prompt social interaction, manage content variety, and optimize session timing.

### G. Clinical Integration

The clinical interface can provide (e.g., through clinical dashboard 222) assessment tools, documentation features, EHR integration, and provider tools, for use by a healthcare user. The assessment tools can include standardized score reporting, trend visualization, risk-level indicators, change-detection alerts, and comparative analysis. The documentation features can include clinical note integration, assessment documentation, treatment planning, progress tracking, and outcome measurement. EHR integration can include bi-directional data exchange, documentation synchronization, order management, results reporting, and alert notification. Provider tools can include a patient monitoring dashboard, population health analytics, risk stratification, care coordination, and quality reporting.

### H. Enterprise Implementation for Senior Care Organizations

The system can include specialized features for implementation in senior care facilities, assisted living communities, and caregiver organizations. These features may include an organization-level dashboard, multiuser management, enhanced analytics, coordination tools, quality metrics, and research optimization.

The organization-level dashboard may provide aggregate population analytics, trend visualization across resident or patient cohorts, resource-utilization optimization, staff-assignment recommendations, and care-quality metrics. The multi-user management pay include centralized account provisioning, role-based access controls, group activity scheduling, bulk content management, and family portal integration. The enhanced analytics may include a neurocognitive-decline early-warning system, intervention-effectiveness tracking, comparative cohort analysis, social-engagement optimization, and behavioral-pattern recognition. The care-coordination tools may include a cross-functional communication platform, task assignment and tracking, intervention documentation, shift-handoff optimization, and care-plan integration. The quality metrics may include resident or patient engagement levels, neurocognitive-activity participation rates, intervention-adherence tracking, satisfaction measurement, and outcome documentation. The resource utilization optimization can include staff-allocation recommendations, intervention prioritization, group-versus-individual activity balancing, time-management optimization, and neurocognitive-decline risk stratification.

The enterprise implementation also enables senior care organizations to identify residents or patients at highest risk for neurocognitive decline, allocate limited resources to those most likely to benefit, document neurocognitive health interventions for quality reporting, provide evidence-based care coordination, engage family members in the care process, and demonstrate quality outcomes to stakeholders.

### I. Security and Privacy Architecture

The system may also implement a security layer 112, which may provide multi-layered security including one or more of the following features and functions. With respect to data protection, the system can implement AES-256 encryption, secure key rotation, data segregation, access logging, and audit trailing. With respect to access control, the system can implement role-based permissions, multi-factor authentication, session management, IP restriction, and device verification. With respect to privacy preservation, the system can implement data minimization, purpose limitation, storage optimization, consent management, and a right to erasure. With respect to compliance features, the system can support HIPAA compliance, GDPR readiness, CCPA compatibility, HITECH alignment, and SOC 2 certification.

### J. High-Level Data Flow

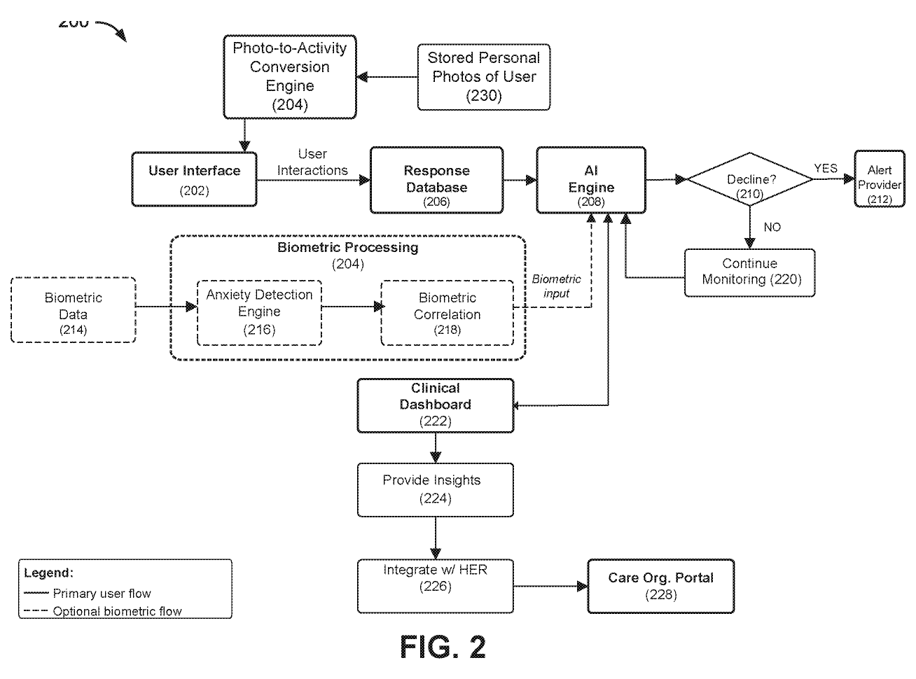

FIG. 2 is a flow diagram showing the main functionalities and components of the NID system introduced here, according to at least some implementations. As shown, the system 200 may include a user interface 202, (optionally) a biometric processing component 204, a response database 206, an AI engine 208, and a clinical dashboard interface 222. In some implementations, all of these components are implemented in/by the mobile application layer 108. In other implementations, one or more of these components (e.g., the AI engine 208 and clinical dashboard interface 222) are implemented in/by one or more remote devices, e.g., one or more servers.

### 1. User Interface

The user interface 202 of the mobile application layer 108 on the mobile device captures and analyzes how users engage with photo-based activities. It implements a high-frequency sampling loop that records all touch interactions, including precise coordinates, timing, pressure, and gesture trajectories. These raw inputs undergo real-time feature extraction that calculates velocity, acceleration, directional changes, and precision metrics. The user interface 202 may employ a dual-path processing architecture with one thread dedicated to maintaining UI responsiveness while a parallel high-priority thread ensures timing precision for neurocognitive measurements. Specialized algorithms can be used to detect and classify interaction patterns including hesitations, error corrections, exploratory behaviors, and confidence indicators. The user interface 202 can adapt to individual interaction styles through a personalization layer that establishes baseline patterns for each user, enabling more precise detection of deviations that might indicate neurocognitive changes.

### 2. Response Database

The response database 206 stores and organizes user interaction data in a structured format optimized for neurocognitive assessment. In some embodiments, the response database 206 captures at least the following three types of information:
1.Interaction Events: Basic user actions (taps, swipes, drags) with timestamps and precision measurements that reveal neurocognitive processing patterns.
2.Session Context: Information about the assessment environment, activity parameters, and user state that provides essential context for interpreting results.
3.Derived Metrics: Calculated values that translate raw interactions into meaningful neurocognitive indicators.

The response database 206 can organize this data to support two functions: detailed analysis of individual sessions and detection of subtle changes over time. It can maintain connections between interactions and the specific photos, activities, and neurocognitive domains they relate to, enabling the system to track performance across different neurocognitive areas. The storage approach balances the need for detailed recent data with efficient long-term storage of historical patterns, ensuring the system can detect meaningful neurocognitive changes while managing data growth over time.

### 3. Biometric Integration (Optional)

The system may utilize a parallel processing architecture that runs biometric analysis alongside neurocognitive scoring. Toward that end, and referring again to FIG. 2, the system 200 may optionally include a biometric processing component 204 (note that any reference to optionality herein is merely for emphasis and does not imply that other components are mandatory or necessary). If/when biometric data 314 is available, the system 200 may integrate physiological data through direct smartwatch connections or health platform APIs. Signal preprocessing algorithms can standardize data formats and sampling rates from diverse sources. Temporal alignment techniques can synchronize physiological measurements with neurocognitive interaction data to millisecond precision. For heart rate analysis, the system can implement frequency-domain processing using Fast Fourier Transform (FFT) to extract heart rate variability (HRV) components indicative of autonomic nervous system state. Temperature data can undergo trend extraction with adaptive filtering to isolate meaningful changes from sensor noise. A biometric correlation engine 218 can employ multivariate regression to quantify relationships between physiological states and neurocognitive performance, while an anxiety detection engine 216 includes one or more machine learning classifiers that identify patterns associated with anxiety, fatigue, and other states affecting neurocognitive testing. Based on detected correlations, compensation algorithms can generate adjustment factors to account for physiological influences on neurocognitive scores.

### IV. Photo-to-Activity Conversion Engine

The mobile application layer 108 may include a photo-to-activity conversion engine 204, which can access a user’s camera roll stored on the mobile device and transform their personal photographs into standardized neurocognitive assessment activities through a sophisticated multi-stage process. The process can begin by prioritizing metadata analysis, as some neurocognitive activities can be generated solely from temporal and location information without requiring deep visual content analysis. Additional processing stages then analyze visual content to enable a wide variety of activity types, from object identification and memory tasks to chronological ordering and spatial puzzles.

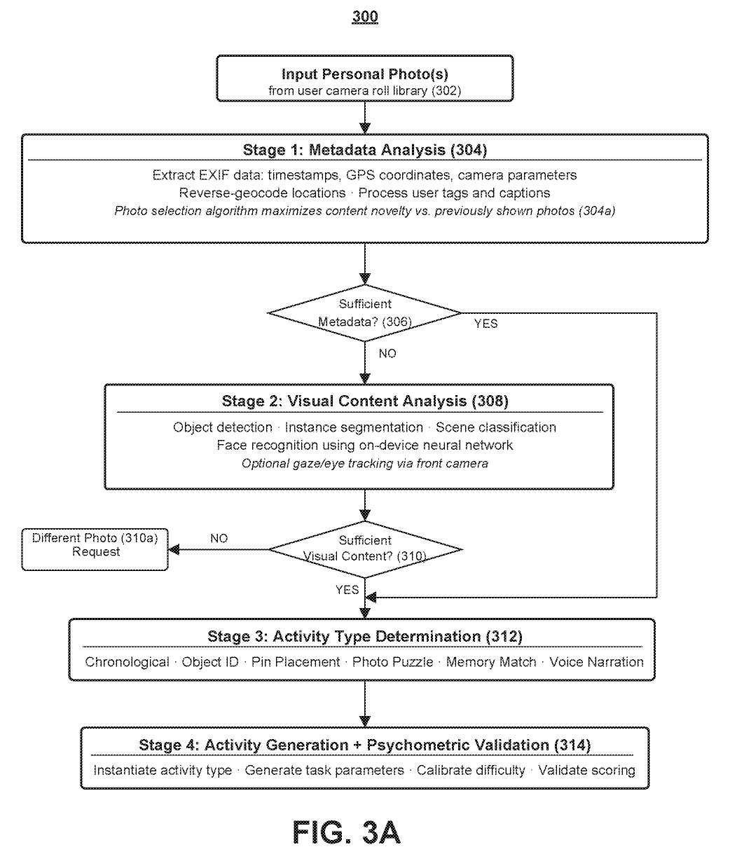

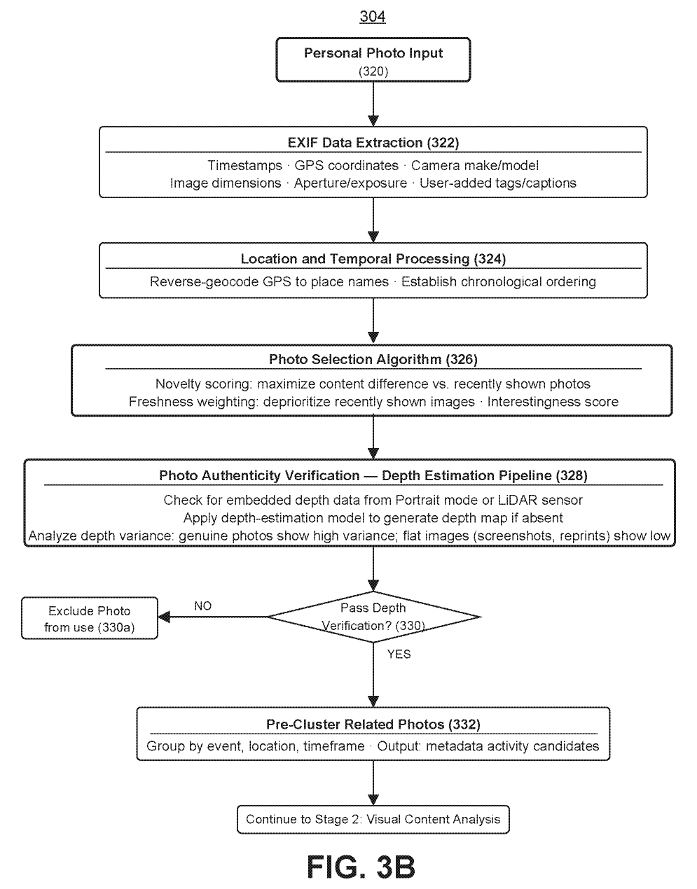

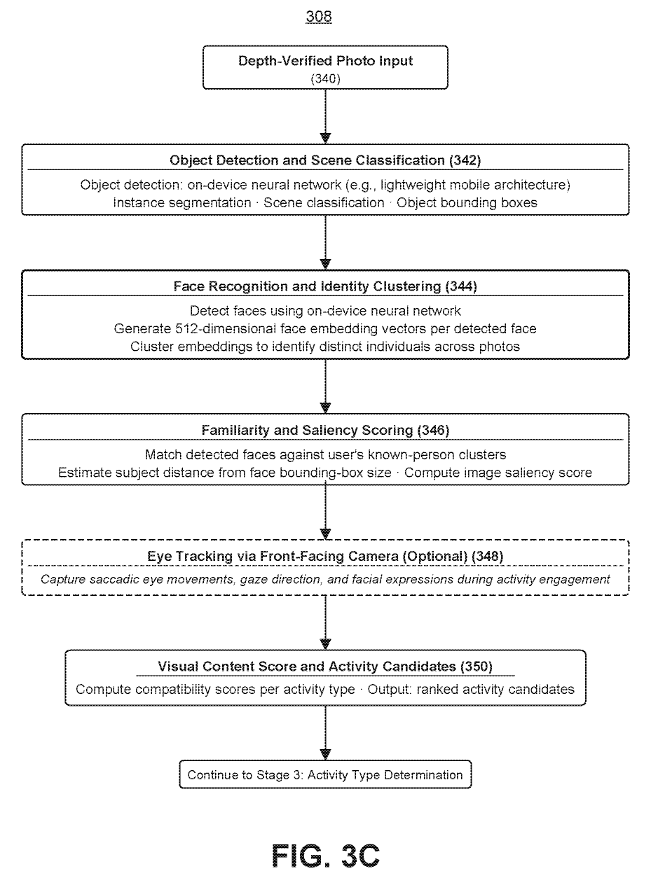

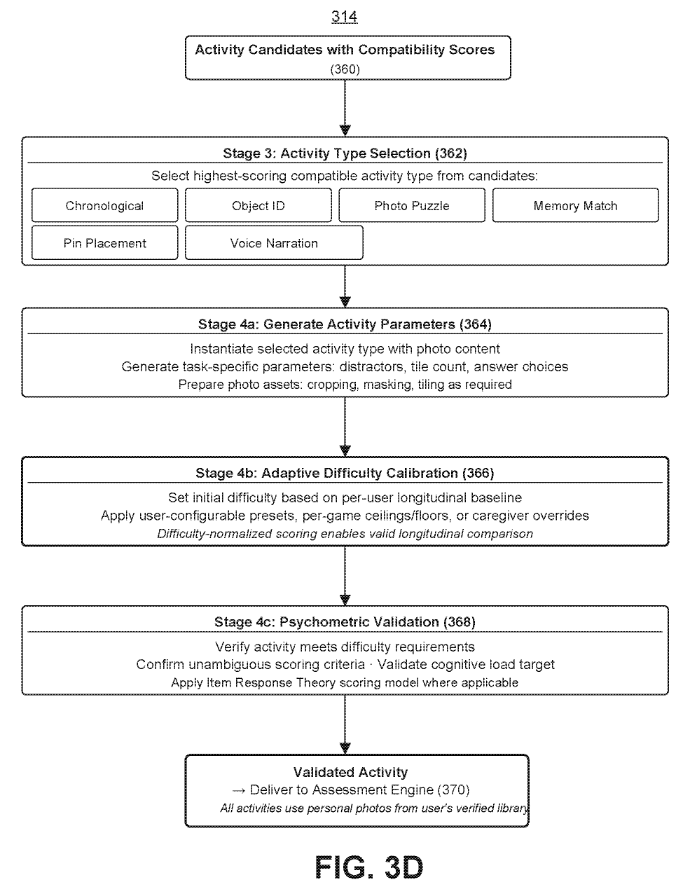

As illustrated in FIG. 3A, the photo-to-activity conversion engine 204 can perform a structured process 300, including first inputting (302) personal photos from a user’s camera roll library, followed by the following four distinct primary stages: 304, 308, 312 and 314.

### A. Metadata Analysis (Primary Entry Point)

In the first stage, metadata analysis 304, extraction algorithms parse EXIF data for timestamp, GPS coordinates, and camera parameters. Location data is reverse-geocoded to meaningful place names using a lightweight on-device databases, captions, and manual groupings are incorporated into the metadata representation. Photos are pre-clustered based on temporal proximity and location similarity. The system then determines at step 306 whether sufficient metadata exists to generate time-based or location-based activities without requiring detailed visual analysis of the photos. Step 306 may be performed on a per-photo basis. In other implementations, it may be performed on groups of photos, or on a combination of groups of photos and individual photos. If it is determined that sufficient metadata exists, then the process proceeds directly to activity type determination in step 312, discussed below. Otherwise, the process proceeds to visual content analysis in step 308.

### B. Visual Content Analysis (Secondary Processing)

For activities requiring visual content understanding, in the second stage, visual content analysis 308, the system may process the image through several distinct stages, including: object detection, instance segmentation, facial recognition, scene classification, and confident scoring. Object detection, for example can use MobileNetV3 with SSD to identify and locate distinct objects within the photo. Instance segmentation can utilize, for example Mask R-CNN to create precise object boundaries for masking or hiding operations. Facial recognition can employ, for example, FaceNet to identify recurring individuals across the photo collection. Scene classification can categorize environments and settings. All detected elements can be assigned confidence scores, bounding boxes, and segmentation masks.

Following the visual content analysis at step 308, the system determines whether sufficient visual content was extracted. If sufficient visual content was extracted, the process proceeds to activity type determination in step 312. Otherwise, the process requests a different photo from the user’s camera roll at step 310a

### C. Activity Type Determination

Based on available metadata and visual content analysis, the system in the activity type determination stage 312 evaluates suitability for various activity types:
1) Chronological ordering tasks (utilizing timestamp metadata)
2) Location-based memory tasks (utilizing GPS and place data)
3) Object identification tasks (utilizing object detection and segmentation)
4) Object location tasks (utilizing bounding box coordinates)
5) Visual puzzles (e.g., photo puzzles, utilizing image segmentation for piece creation)
6) Photo completion tasks (utilizing inpainting capabilities.

For each activity type, a compatibility score can be calculated based on available data quality and quantity.

### D. Activity Generation and Psychometric Validation

In the activity generation stage & psychometric validation stage 314, the system instantiates the selected activity type with specific content:
1) For chronological tasks: Selecting photo sets with clear temporal progression
2) For object identification: Selecting photos with high-confidence detected objects, generating masks to hide them.
3) For location tasks: Creating pin-placement activities using object bounding box coordinates.
4) For puzzles: Algorithmically determining optimal segmentation into pieces based on visual complexity. Task instructions can be generated with contextual references to the specific photo content. Difficulty parameters can be calibrated based on user history and neurocognitive profile.

The generated activity undergoes validation checks to ensure:
1) Appropriate difficulty level based on user history
2) Clear correct answer(s) with unambiguous scoring criteria
3) Balance between engagement and assessment value
4) Appropriate neurocognitive load for targeted domains
5) If validation fails, parameters are adjusted or an alternative activity type is selected

### E. Example Games/Activities

1. Chronological Ordering

In a chronological-ordering activity, the system may identify a set of, for example, five photos from a vacation spanning three days and present the photos in random order at the bottom of the screen. The user may then drag and arrange the photos in the correct chronological sequence. Difficulty may vary such that a novice mode uses photos from different days with distinct visual cues, whereas an advanced mode uses photos from the same day with subtle temporal progression. This activity can target episodic memory, temporal sequencing, and executive function.

2. Object Identification

In an object-identification activity, the system may identify multiple distinct objects in a photo -- such as a dog, a ball, a bench, and a tree -- and may apply an object-segmentation mask to hide one object, such as the ball. The user may then be asked, “What object is missing from this picture?” Difficulty may vary based on the size and centrality of the hidden object, with peripheral or smaller objects creating higher difficulty. This activity can target visual memory, object permanence, and attention to detail.

3. Pin Placement

In a pin-placement activity, the system may detect and locate multiple objects in a complex scene -- such as a picnic with food items, people, and animals -- and may create an instruction directing the user to locate a specific object, such as “Place the pin on the rabbit.” The user may then tap the precise location of the specified object. Difficulty may vary based on the size of the target object, the visual complexity of the surroundings, and the similarity of distractor objects. This activity can target visual search, selective attention, and object recognition.

4. Photo Puzzle Activity

In a photo-puzzle activity, the system may analyze visual complexity and natural segmentation boundaries, divide the photo into puzzle pieces -- for example four to twelve pieces depending on difficulty -- and require the user to reconstruct the original photo by dragging and arranging the pieces. Difficulty may vary based on the number of pieces, the complexity of the image, and the similarity between pieces. This activity can target visuospatial processing, pattern recognition, and executive function.

### F. Photo-to-Activity Conversion Processing Flow

FIG. 3B shows an example of the processing flow of the metadata analysis stage 304 of the photo-to-activity conversion process of FIG. 3A. This stage serves as a primary entry point for qualifying personal photos for use in neurocognitive activities. The process 304 begins with personal photo input 320, where one or more digital images are retrieved from a user’s camera roll or photo library.

The system then at step 322 performs EXIF data extraction to parse technical metadata embedded in the image files. The extracted metadata may include, for example, timestamps, GPS coordinates, camera make and model, image dimensions, aperture and exposure settings, and any user-added tags or captions. These data points provide initial context for the photograph without requiring computationally intensive visual analysis.

Following extraction, the system performs location and temporal processing at step 324. This may include reverse-geocoding GPS coordinates to place names using a database and establishing a chronological ordering of the photos to facilitate time-based neurocognitive tasks.

A photo selection algorithm then evaluates the candidate photos at step 326 to ensure variety and engagement. The algorithm calculates a novelty score to maximize content difference compared to recently shown photos, applies freshness weighting to deprioritize images used in recent sessions, and computes an interestingness score based on metadata richness and image parameters.

Next, to ensure the integrity of the assessment, the system performs a photo authenticity verification depth estimation pipeline at step 328. This pipeline verifies that each photo represents a genuine camera-captured three-dimensional scene rather than a screenshot or a photo of a printed image. The pipeline may check for embedded hardware depth data (such as LiDAR or portrait mode data) or apply an AI depth-estimation model to generate a synthetic depth map. The system then analyzes depth variance; genuine photos typically exhibit high depth variance, while flat images (e.g., screenshots) show low variance.

Based on this analysis, the system reaches a decision point 330 to determine if the photo passes depth verification. If the photo fails this check, it is excluded from use. If the photo passes, the system proceeds to pre-cluster related photos at step 332, grouping them by event, location, or timeframe. Finally, at step 334 the process outputs to the next stage the metadata-qualified activity candidates and continues to Stage 2: Visual Content Analysis (308).

FIG. 3C shows an example of the processing flow of the visual content analysis stage 308 of the photo-to-activity conversion process of FIG. 3A. This stage involves deep analysis of the image pixels to extract semantic meaning and identify potential subjects for neurocognitive tasks.

The process begins at step 340, where depth-verified photo input is received. This input comprises the set of personal photographs that have successfully passed the metadata analysis and authenticity verification steps described previously.

At step 342, the system performs object detection and scene classification. In at least some embodiments, this step utilizes an on-device neural network (e.g., a lightweight mobile architecture optimized for mobile computing devices) to perform instance segmentation and scene classification. The system may further generate object bounding boxes to define the spatial boundaries of detected items within the image, which can later be used to generate "Object ID" or "Pin Placement" activities.

The process then proceeds to face recognition and identity clustering at step 344. Here, the system detects faces using an on-device neural network and generates high-dimensional face embedding vectors (e.g., 512-dimensional vectors) for each detected face. These embeddings are clustered to identify distinct individuals across the user's photo library, allowing the system to distinguish between a "frequent" contact (such as a spouse) and a "rare" contact (such as a distant acquaintance).

Next, at step 346, the system performs familiarity and saliency scoring. Here the system may match detected faces against the user's known-person clusters to determine the biographical relevance of the subjects. Additionally, the system may estimate the subject's distance from the camera based on face bounding-box size and compute an image saliency score to identify the most visually interesting or important regions of the photograph.

In some embodiments, as illustrated in the optional step 348, the system may also perform eye tracking via the front-facing camera of the mobile device. This step may involve capturing saccadic eye movements, gaze direction, and facial expressions of the user in real-time during engagement with a generated activity, providing additional multi-modal biomarkers for neurocognitive assessment.

At step 350, the system computes a visual content score and identifies activity candidates. This may involve calculating compatibility scores for different activity types (e.g., chronological, memory match) based on the specific content found in the photo. The system then outputs to the next stage a ranked list of activity candidates that are most likely to provide an engaging and valid neurocognitive challenge.

Finally, at step 352, the process continues to Stage 3 for the final determination of the activity type to be presented to the user.

FIG. 3D shows an example of the processing flow of the activity generation and psychometric validation stage 314 of the photo-to-activity conversion process of FIG. 3A. This stage ensures that the activities presented to the user are not only personalized and engaging but also scientifically valid for longitudinal neurocognitive monitoring.

The process begins at step 360, by inputting the ranked list of activity candidates along with their respective compatibility scores. These candidates are the output of the visual content analysis stage 308 and represent potential tasks (e.g., memory match, chronological ordering) that the identified photo content can support.

At step 362, the system performs activity type selection. In this step, the system identifies the highest-scoring compatible activity type from the available candidates, which may include chronological ordering, object identification, pin placement, voice narration, photo puzzles and/or memory match games. This selection is based on the richness of the extracted metadata and visual features relative to the neurocognitive domain being assessed.

Next, at step 364, the system generates activity parameters. This may involve instantiating the selected activity type using the specific personal photo content. The system generates task-specific parameters such as the number and type of distractors, tile counts for puzzles, and specific answer choices for multiple-choice components. During this step, the system may also prepare the necessary photo assets by performing operations such as cropping, masking, or tiling as required by the activity design.

The process then proceeds to step 366, where the system performs adaptive difficulty calibration. The system sets an initial difficulty level for the activity based on the user's longitudinal baseline. This calibration may also incorporate user-configurable presets, per-game ceilings or floors, or specific caregiver overrides. By utilizing difficulty-normalized scoring, the system enables performance data to be validly compared across different activities and sessions over time.

At step 368, the system performs psychometric validation on the generated activity. This validation enables the activity to meet predefined difficulty requirements and possesses unambiguous scoring criteria. The system may also validate the neurocognitive load target to ensure it is appropriate for the intended assessment and may apply an Item Response Theory (IRT) scoring model to further refine the assessment's precision.

Finally, at step 370, the validated activity is delivered to the assessment engine for administration to the user. All such activities utilize personal photographs from the user’s verified library to maximize autobiographical memory activation and user engagement.

### V. Clinical Dashboard

Referring again to FIG. 2, the clinical dashboard interface 222 transforms neurocognitive assessment data into clinically meaningful visualizations and insights. It provides healthcare professionals with a comprehensive view of a patient's neurocognitive health metrics and longitudinal trends. Raw performance data undergoes domain-specific processing to extract metrics aligned with standardized neuropsychological assessments. Statistical summarization calculates key indicators including mean scores, variability measures, trend gradients, and comparison against normative populations. The clinical dashboard interface 222 includes a visualization engine that creates interactive graphical representations optimized for clinical interpretation, including domain-specific performance charts, longitudinal trends with confidence intervals, and comparative visualizations. The clinical dashboard interface 222 may further include population analytics components. For senior care implementations, the population analytics components may provide risk stratification, intervention prioritization, and resource optimization tools. The system implements role-based transformations that present different levels of detail based on the clinical role of the viewer, from detailed metrics for specialists to simplified status indicators for caregivers. Alert management components prioritize and route notifications based on clinical significance and provider workflows.

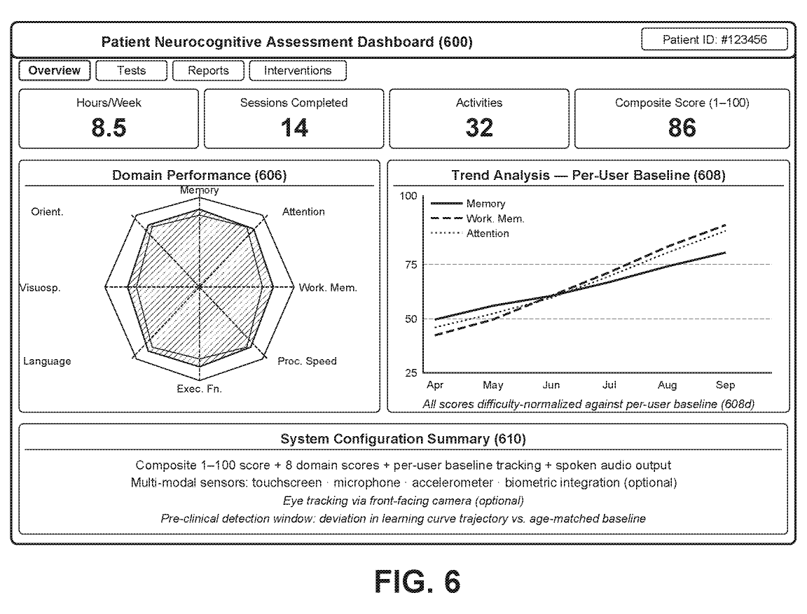

FIG. 6 illustrates an example of a dashboard display that may be generated by the clinical dashboard interface 222, which provides healthcare professionals with a comprehensive view of a patient's neurocognitive health metrics and longitudinal trends. For example, for a patient having ID #123456, the dashboard displays engagement statistics including 8.5 hours spent on the games/activities in the past week, 14 quizzes completed, 32 activities completed, and an average activity score of 86%. The left visualization panel presents a radar chart of Neurocognitive Domain Performance across six key areas: Memory, Attention, Working Memory, Processing Speed, Executive Function, and Language, allowing clinicians to quickly identify relative strengths and weaknesses across neurocognitive domains. The right panel features a Trend Analysis graph tracking performance in Memory, Working Memory, and Attention over a seven-month period (April through October), with color-coded trend lines and status indicators showing stable Memory performance (orange) and improving trends in Working Memory and Attention (blue and teal). This visualization enables healthcare providers identify subtle changes over time, recognize domain-specific patterns, and make data-driven clinical decisions about neurocognitive health interventions and treatment plans.

### VI. AI Engine / Neurocognitive Decline Detection

The AI processing layer 110 (FIG. 1B) includes an AI engine 208 (FIG. 2). The purpose and function of the AI engine 208 is to make (or alternatively, to facilitate) a decision 210 on whether neurocognitive decline is detected. The AI engine 208 may be implemented as one or more discrete AI components (e.g., programs, algorithms and/or machine-learning models). The functionalities of the one or more components that make up the AI engine 208 may span multiple levels of analysis, from deeply personal, user-specific processing at one end of a spectrum, through clinically oriented risk and alert generation for caregivers and practitioners, to aggregated insights across user populations within and across organizations at the other end of the spectrum.

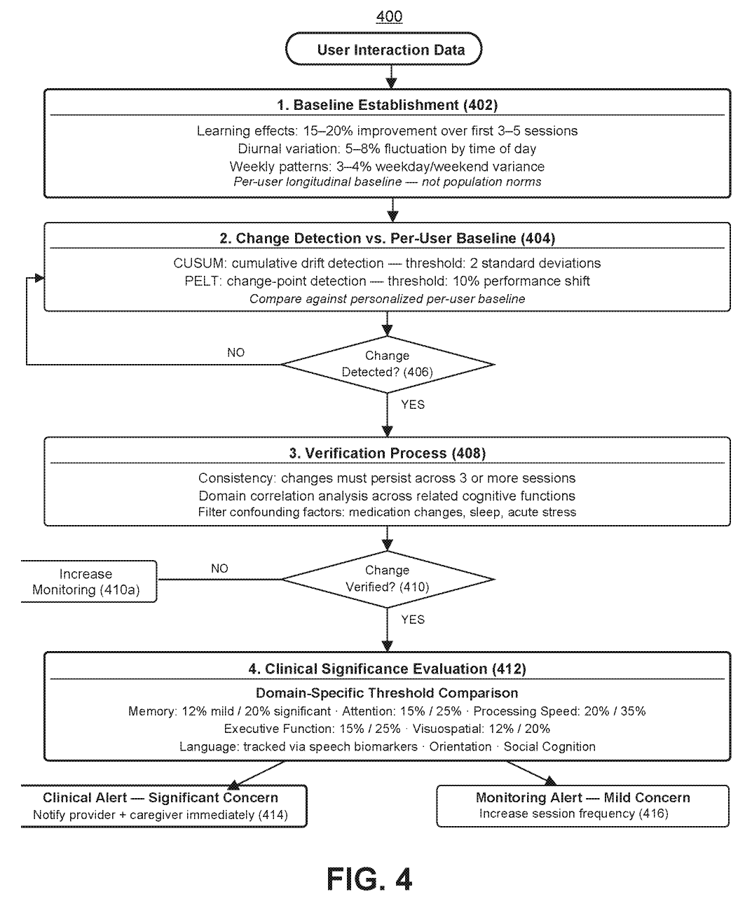

As shown in FIG. 4, the AI engine 208 may perform a multi-stage process 400 of neurocognitive decline detection for each user, by distinguishing pathological neurocognitive changes from normal variability. FIG. 4 illustrates how user interaction data progresses through a baseline comparison step 402, a change detection step 404, a change verification step 408, and a classification step 412, before generating appropriate clinical alerts. This structured approach reduces false positives while maintaining sensitivity to meaningful neurocognitive changes.

The process begins with baseline establishment in step 402. In this step the system creates personalized reference points using statistical techniques that specifically account for, for example, learning effects (e.g., typical 15-20% improvement over first 3-5 sessions), diurnal variations (e.g., documented 5-8% performance fluctuation by time of day), weekly patterns (e.g., observed 3-4% variance between weekday/weekend performance), and gradual age-related changes (e.g., expected 1-2% annual decline in processing speed for healthy aging).

Next, in step 404 the system performs change detection (i.e., detection of potential neurocognitive decline) by comparing current performance against established baselines. This comparison may use, for example, Cumulative Sum (CUSUM), which tracks small changes that add up over time. A change may be detected when these changes reach twice what is normally expected, for example. This comparison may also use, for example, Pruned Exact Linear Time (PELT), which finds exactly when a significant change happened. The system may note a change where performance shifts by at least 10%, for example.

If decision step 406 determines that no change was detected in step 404, the process continues monitoring by repeating step 404. If a change was detected at step 404, the process proceeds to step 408, which is the verification process, in which the system applies additional validation criteria, such as: a consistency requirement (e.g., changes must persist across at least three sessions; domain correlation analysis (e.g. examines related neurocognitive functions for corroborating changes); and confounding factor filtering (e.g., accounts for medication changes, sleep quality, and stress levels).

If a change was not verified in step 408, the process increases monitoring at step 410a. The increased monitoring may include prompting the user to complete their daily challenges consistently without skipping sessions, so the system can gather enough data to know whether what it saw was a real change in neurocognitive function or just noise. The system may stay in this closer-watch mode until it either gathers enough evidence to confirm something meaningful is happening, or enough sessions pass without further signal and it relaxes back to normal.

If a change was verified at step 408, the process proceeds to step 412, which is clinical significance evaluation. In this step the system categorizes detected changes by comparison to known progression patterns from clinical datasets across specific neurocognitive domains. Specifically, one or more pattern recognition algorithms differentiate between global neurocognitive changes affecting multiple domains, domain-specific changes limited to particular neurocognitive functions, and transient vs. persistent patterns based on duration and stability.

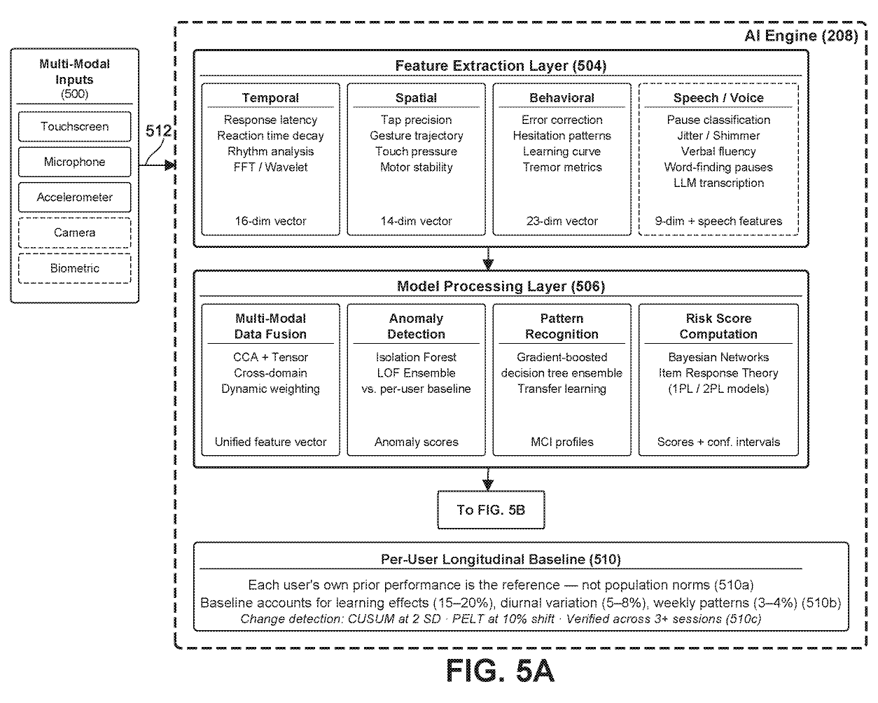

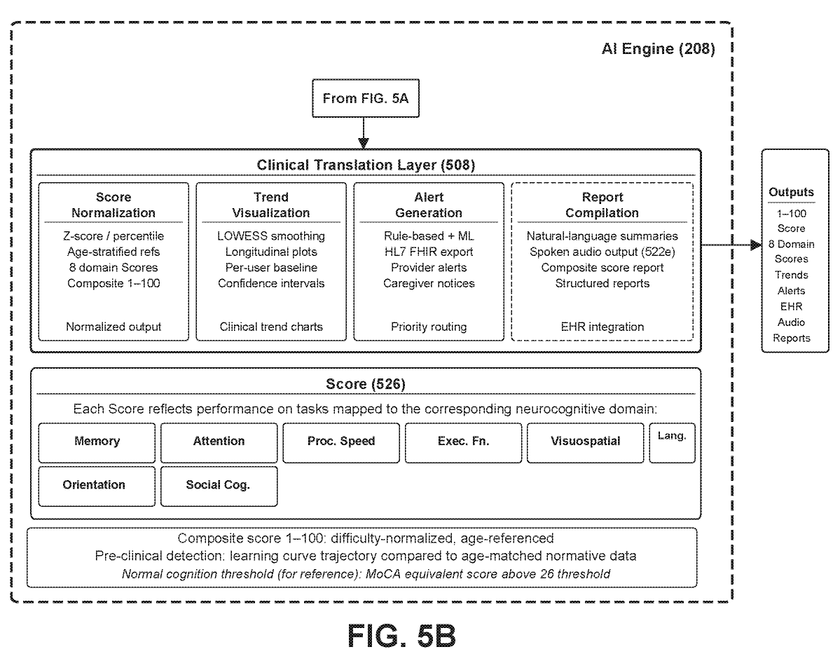

In certain embodiments, as further illustrated in FIGS. 5A and 5B, the AI engine 208 may include and process user interaction data through three interconnected software layers, for example: a feature extraction layer 504, a model processing layer 506, and a clinical translation layer 508. This layered architecture enables raw interaction data to be progressively refined into clinically actionable insights. Each layer can employ specialized algorithms optimized for specific aspects of neurocognitive assessment, with outputs from each layer feeding into subsequent processing. This architecture enables the system to identify subtle neurocognitive patterns while providing interpretable results that can inform clinical decision-making.

### A. Feature Extraction Layer

In some embodiments, the feature extraction layer 504 may extract the following four distinct categories of features from raw interaction data: Temporal feature computation, spatial pattern analysis, behavioral marker extraction, and error pattern identification. Temporal feature computation calculates timing-based metrics, including response latency distributions, inter-action intervals, processing-speed indicators, and time-based learning curves across repeated stimuli. Example algorithms may include Fast Fourier Transform (FFT) and wavelet decomposition for frequency analysis of interaction patterns. Example specific features may include response latency distributions, including mean, median, variance, skewness, and kurtosis; initiation delays; completion times; rhythm-consistency metrics; inter-tap intervals; and response-time decay rates for repeated stimuli. Example inputs may include raw timestamp sequences from user interactions, such as press, drag, and hold events, calibrated to millisecond precision, and example outputs may include a 16-dimensional vector of temporal features with normalized scores.

Spatial pattern analysis processes interaction-positioning data to derive touch-precision metrics, gesture-trajectory smoothness, spatial-memory accuracy, and screen-navigation efficiency patterns. Example algorithms may include density-based spatial clustering (DBSCAN) and trajectory analysis using Fréchet-distance calculations. Example specific features may include touch precision, such as deviation from targets; gesture smoothness; path-efficiency ratios; screen-coverage patterns; spatial-memory accuracy; and fine-motor-control stability metrics. Example inputs may include coordinate sequences (x,y) with pressure values from touch events, and example outputs may include a 14-dimensional vector of spatial interaction features.

Behavioral marker extraction identifies interaction behaviors, including error-correction patterns, task-switching efficiency, exploration tendencies, and adaptation rates to new stimuli. Example algorithms may include Hidden Markov Models (HMMs) for sequence-pattern recognition and k-means clustering for behavior classification. Example specific features may include error-correction patterns, hesitation frequencies, exploration strategies, perseveration tendencies, distractibility indicators, and learning-curve characteristics. Example inputs may include combined temporal-spatial interaction sequences with task-context metadata, and example outputs may include a 23-dimensional behavioral-pattern vector with abnormality scores.

Error-pattern identification analyzes mistakes and corrections to extract error frequency and typology, perseveration tendencies, self-correction capabilities, and error distribution across neurocognitive domains. Example algorithms may include decision-tree-based feature-importance analysis and change-point detection. Example specific features may include task-switching costs, inhibition-control metrics, planning efficiency, working-memory capacity, and neurocognitive-flexibility indicators. Example inputs may include performance across tasks requiring executive control, sequencing, and multi-step processing, and example outputs may include a 9-dimensional error-pattern vector with domain-specific scores.

### B. Model Processing Layer

The model processing layer 506 of the AI engine 208 processes extracted features through several analytical components, including: 1) multi-modal data fusion, 2) anomaly detection, 3) pattern recognition, and 4) risk score computation.

1. Multi-modal data fusion

Multi-modal data fusion integrates diverse feature types into unified representations while preserving domain-specific information. Example algorithms may include Canonical Correlation Analysis (CCA) and tensor-fusion networks. The processing may align and normalize heterogeneous feature vectors from different neurocognitive domains through a multi-step approach, including dimensional alignment, scale normalization, cross domain correlation mapping and waited integration.

Dimensional alignment transforms features with different dimensionalities into a common representation space, such as projecting 16-dimensional temporal and 14-dimensional spatial features into a shared 20-dimensional latent space. Scale normalization applies domain-specific transformations, such as log-transformation for response times and min-max scaling for spatial metrics. Cross-domain correlation mapping preserves relationships between domains, such as preserving relationships between timing variability and touch-precision degradation. Weighted integration assigns dynamic weights to different domains based on measurement reliability and task relevance. Example inputs may include feature vectors from all extraction components with varying dimensionality, and example outputs may include a unified representation vector with preserved cross-modal relationships.

2. Anomaly detection

Anomaly detection identifies deviations from both personal baselines and normative patterns that may indicate neurocognitive changes. Example algorithms may include Isolation Forest and Local Outlier Factor (LOF) with ensemble voting. The model training may use, for example, a semi-supervised approach with labeled clinical data (e.g., 10%) and unlabeled interaction data (e.g., 90%). The processing may identify statistically significant deviations from both personal baselines and population norms. Example inputs may include fused feature vectors and historical performance distributions, and example outputs may include anomaly scores with confidence intervals and domain-specific flags.

3. Pattern recognition

Pattern recognition classifies interaction patterns using supervised learning models trained on clinically validated datasets to associate patterns with neurocognitive states. Example algorithms may include an ensemble of gradient-boosted decision trees (XGBoost) and specialized convolutional neural networks. Model training may use a transfer-learning approach starting with pre-trained models on clinical datasets and fine-tuning on application-specific data.

The processing may classify interaction patterns into known neurocognitive profiles and detect signature patterns of specific conditions. For example, MCI pattern detection may identify preserved procedural memory, declining episodic memory, increased reliance on semantic knowledge, and normal initial response times with rapidly increasing latency during extended sessions, such as a 78% similarity to an amnestic MCI reference. As another example, a depression-related neurocognitive profile may distinguish global slowing, pronounced diurnal effects, effort-dependent performance, and temporal consistency, such as a 67% match to mood-related patterns versus a 32% match to neurodegenerative patterns. Example inputs may include multi-dimensional feature representations from the data-fusion component, and example outputs may include neurocognitive-profile classifications with probability distributions across possible conditions.

4. Risk-score computation

Risk-score computation calculates domain-specific neurocognitive-risk indicators with confidence intervals based on recognized patterns and anomalies. Example algorithms may include Bayesian networks with expert-informed prior probabilities and adaptive weighting. Model training may combine supervised learning with knowledge-based rules derived from clinical literature. Example inputs may include anomaly detections, trend analyses, pattern classifications, and demographic context, and example outputs may include clinical risk scores across neurocognitive domains with uncertainty quantification. The processing may integrate outputs from all model components to calculate domain-specific and overall neurocognitive-risk scores. For example, for a 72-year-old user, the system may compute Memory (37/100), Attention (22/100), Processing Speed (65/100), Executive Function (28/100), and Overall Risk (42/100). As another example, for an 83-year-old with variable engagement, the system may produce confidence-weighted assessments such as Memory at 58/100 with 72% confidence and Overall Risk at 61/100 with 79% confidence.

### C. Clinical Translation

The clinical translation layer 508 of the AI engine 208 transforms analytical results into clinically meaningful outputs. In certain implementations it includes four major functions: score normalization, trend visualization, alert generation, and report compilation.

1. Score normalization

Score normalization converts raw analytical measures to standardized scales comparable to established clinical assessments. Example algorithms may include non-parametric percentile ranking and z-score calculations with age-stratified references. The processing may convert raw performance metrics to clinically interpretable scales comparable to standard assessments. Example inputs may include raw performance scores and demographic information, and example outputs may include normalized scores on scales equivalent to MMSE, MoCA, and domain-specific neuropsychological tests. The output produced by the clinical translation layer 508 for any given individual may include multiple scores 526, including a difficulty-normalized composite neurocognitive health score, for example on a scale of 0 to 100, and multiple domain scores related to the user of the mobile computing device. In some embodiments, the domain scores include a separate score for each of eight MoCA domains, namely memory, attention, processing speed, executive function, visuospatial ability, language, orientation, and social cognition. Further, each of those eight domains encompasses multiple subdomains, and a separate score may be computed by the system for each subdomain. The individual scores for any two or more subdomains within a domain may be used to compute the score for the domain that encompasses those subdomains.

2. Trend visualization

Trend visualization generates interpretable visualizations of neurocognitive trajectories designed for clinical review. Example algorithms may include LOWESS / LOES (STL) and Gaussian-process visualization. Model training may use, for example, supervised learning on longitudinal neurocognitive-assessment datasets with known outcomes. Example inputs may include longitudinal assessment data with trend-analysis results, and example outputs may include interactive visualization components with clinically relevant annotations. The processing may separate long-term trajectories from short-term fluctuations and periodic patterns. For example, memory-recall performance showing a 10-week trend of 82%, 79%, 85%, 80%, 78%, 76%, 75%, 73%, 72%, and 70% may be decomposed into a declining trajectory of -1.3% per week, weekend-improvement patterns of 3-4%, and random fluctuations of plus or minus 2.5%. As another example, response analysis over eight weeks showing increasing variability from 342 ms plus or minus 45 ms to 389 ms plus or minus 89 ms may reveal not only an increasing mean of 6 ms per week but also faster-growing variability, suggesting declining neurocognitive consistency. The processing may further generate interpretable visualizations of neurocognitive trajectories optimized for clinical understanding.

3. Alert generation

Alert generation creates priority-weighted notifications when significant neurocognitive changes are detected. Example algorithms may include a rule-based expert system with severity classification using random-forest models. Example processing may determine clinical significance of detected changes and appropriate notification urgency. Example inputs may include anomaly scores, trend analyses, and risk computations with confidence metrics. Example outputs may include prioritized clinical alerts with supporting evidence and recommendation pointers delivered to healthcare providers, caregivers, and users. For healthcare providers, alerts may be transmitted securely through integration with electronic health record systems via HL7 FHIR-compliant protocols, secure provider-portal notifications with role-based access control, encrypted email notifications for urgent alerts depending on severity, and SMS or text-message notifications for critical alerts requiring opt-in. For caregivers, modified alerts with appropriate privacy controls may be delivered via a dedicated caregiver mobile application with push notifications, a web portal with customizable alert thresholds, and email digests summarizing trends weekly or monthly. For users, appropriate non-alarming feedback may be provided through in-app notifications encouraging continued engagement, activity recommendations based on detected patterns, and optional progress summaries with positive framing.

4. Report compilation

Report compilation assembles comprehensive documentation of findings with supporting evidence for clinical review. Example algorithms may include natural-language generation using template-based approaches and context-aware text generation. Example processing may create comprehensive clinical summaries in standardized formats compatible with medical documentation. Example inputs may include all assessment results, analyses, and visualizations, and example outputs may include structured clinical reports with key findings, supporting data, and longitudinal context.

### D. Per-User Longitudinal Baseline 510

The system may establish and maintain a per-user longitudinal baseline derived only from each individual user's own prior performance history, rather than by comparing against population norms or age-matched cohort data. The baseline can be dynamically adjusted to account for known sources of performance variability, such as learning effects of approximately 15–20% observed over the first 3–5 sessions, diurnal variation of approximately 5–8% reflecting time-of-day differences in neurocognitive performance, and weekly patterns of approximately 3–4% reflecting systematic differences between weekday and weekend performance. For example, genuine change may be identified only when performance deviations exceed the user's personal baseline by a specified amount (e.g., by two standard deviations) as measured by a cumulative sum algorithm or when an abrupt shift exceeding a certain percentage (e.g., 10%) is detected by a change-point detection algorithm -- and in either case, only after the detected change is confirmed across a minimum number (e.g., three) of successive assessment sessions.

### VII. Biometric Processing

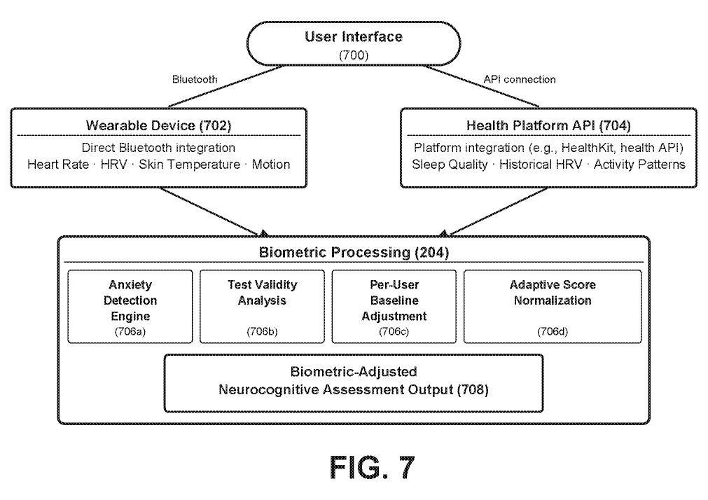

As noted above, biometric processing, as implemented by biometric processing component 204, is may execute in parallel with neurocognitive analysis. FIG. 7 illustrates an example of the functionality of the biometric processing component 204 in greater detail. The biometric processing component 204 in at least some embodiments includes an anxiety detection engine 706a, a test validity analysis module 706b, per user baseline adjustment module 706c, and adaptive score normalization module 706d. The inputs to the biometric processing component 204 may come from one or more wearable devices 702 and/or from one or more health platform application programming interfaces (APIs) 704.

The anxiety detection engine 706a may represent the anxiety detection engine 216 in FIG. 2. Its inputs may include, for example: heart rate data, heart rate variability metrics (e.g., RMSSD, SDNN, pNN50) and frequency domain components (LF/HF ratio); skin temperature readings with baseline-normalized values; optional motion data from accelerometer indicating restlessness or tremor; and/or historical anxiety pattern data for the individual when available. Its outputs may include, for example: binary anxiety state classification (present/absent) with confidence score; continuous anxiety intensity estimation; anxiety pattern characterization (acute onset, sustained, fluctuating); contributing factor analysis identifying primary physiological indicators; and/or time-series of anxiety scores for tracking during assessment sessions.

The anxiety detection engine 706a can employ a multi-modal analysis approach using, for example, a Random Forest classifier trained on labeled anxiety episodes from clinical datasets. It may process raw biometric signals into one or more of the following distinct anxiety-related features:
1) Heart rate features: resting rate, acceleration, variability indices (RMSSD, pNN50, SDNN), frequency domain measures (LF/HF ratio, VLF power), reactivity metrics, recovery rates.
2) Skin temperature features: baseline temperature, gradient of change, fluctuation frequency, bilateral comparison (when available), cooling rate, recovery patterns, characteristic anxiety signatures.
3) Motion features: micro-tremor frequency spectra, movement entropy, fidgeting patterns, posture shifts, stillness durations, gesture interruptions, characteristic anxiety-related movement signatures.
4) Contextual features: time of day effects, activity context, previous anxiety episodes, stimuli exposure correlation, neurocognitive load estimation.

The anxiety detection engine 706a may utilize a dual-threshold system with different sensitivity levels, e.g., an initial detection threshold optimized for high sensitivity with moderate specificity, and a confirmation threshold with stricter criteria requiring multiple concordant signals, optimized for lower specificity while maintaining higher sensitivity. A threshold calibration methodology may use ROC curve analysis with weighting to prioritize reduction of false negatives (missed anxiety).

The anxiety detection engine 706a may apply personalized baseline normalization through collection of a specified number of days of baseline data before personalization. It may calculate individual-specific normal ranges for each physiological parameter, including:
1) Time-of-day specific references using 3-hour windows
2) Activity-state specific baselines (rest, light activity, neurocognitive engagement)
3) Exponential weighting of recent data (λ=0.85) to adapt to gradual physiological changes

The anxiety detection engine 706a may implement temporal pattern analysis using, for example, sliding window (e.g., 30-second) calculations with 50% overlap:
1) Primary analysis window (e.g., 30 seconds) to capture acute changes
2) Medium-term windows (e.g., 2 minutes, 5 minutes) to detect sustained patterns
3) Comparative analysis between window lengths to identify characteristic anxiety signatures (rapid onset vs. gradual build)
4) Overlap design (e.g., 50%) to ensure continuous monitoring without transition artifacts
5) Pattern detection algorithms to identify specific anxiety signatures: acute spikes, plateaued elevation, oscillatory patterns, and anticipatory responses

Hence, the anxiety detection engine 706a enables reliable detection of anxiety states during neurocognitive assessment sessions while distinguishing between normal physiological responses to neurocognitive challenge and actual anxiety that might invalidate assessment results.

The test validity analysis component 706b models relationships between physiological states and test performance. It may do so using a Bayesian network, for example. The test validity analysis component 706b may implement a validity scoring algorithm based on research-established thresholds for physiological interference. It further may employ statistical comparison between current physiological metrics and known valid test conditions. Additionally, the test validity analysis component 706b may apply domain-specific validity rules that consider different physiological impacts across neurocognitive domains. It may further perform confidence calculation (e.g., using Monte Carlo simulation) to account for measurement uncertainties.

The inputs to the test validity analysis component 706b may include, for example: anxiety scores and classifications from the detection engine; current physiological metrics compared against established valid testing ranges; neurocognitive domain(s) being assessed in current test components; historical performance-physiology relationships for the individual; and/or research-based validity thresholds for each biometric parameter.

The outputs of the test validity analysis component 706b may include, for example: an overall test validity score (e.g., 0-100%); domain-specific validity scores for different neurocognitive components; specific validity concern flags with severity ratings; recommendations for validity improvement; and/or confidence intervals for validity estimates.

The per-user baseline establishment component 706c implements an adaptive weighted averaging system that calculates personalized physiological baselines. It may utilize contextual filtering to separate rest, light activity, and neurocognitive effort states. It may also apply time-weighted exponential forgetting to emphasize recent patterns while maintaining historical context. It further may implement circadian rhythm awareness that accounts for time-of-day effects on physiological parameters, and may employ anomaly detection and exclusion for baseline calculations to prevent skewing from atypical episodes.

The inputs of the per-user baseline establishment component 706c may include, for example: historical physiological data across multiple days/sessions; context annotations indicating activity levels and assessment states; time-of-day information for circadian adjustment; data quality metrics for weighting contribution to baseline; and/or prior baseline calculations for incremental updating. The outputs of the per-user baseline establishment component 706c may include, for example: personalized baseline values for each physiological parameter (e.g., heart rate, HRV metrics, temperature); context-specific baselines (resting, light activity, during assessment); confidence intervals for baseline estimates; baseline stability metrics indicating reliability; and/or circadian variation profiles for anticipating time-of-day effects.

The adaptive score adjustment component 706d quantifies relationships between physiological states and neurocognitive performance, by using for example, multivariate regression models. It may implement domain-specific adjustment calculators with different parameters for different domains, such as memory, attention, executive function, etc. It may utilize gradient-based optimization to determine minimal sufficient adjustments. It further may apply confidence-weighted scaling that modulates adjustment magnitude based on physiological data reliability. Additionally, the adaptive score adjustment component 706d may implement a two-pass calculation with initial estimation followed by constraint application to prevent over-adjustment.

The inputs of the adaptive score adjustment component 706d may include, for example: raw neurocognitive assessment scores from traditional scoring algorithm; current physiological state characterization including anxiety level; test validity scores for the current assessment; established correlations between physiological parameters and performance metrics; and/or historical adjustment effectiveness data. The outputs of the adaptive score adjustment component 706d may include, for example: adjusted neurocognitive scores with physiological context considered; adjustment rationale with specific contributing factors; confidence intervals for adjusted scores; comparative presentation of raw and adjusted scores; and/or recommendation flags for interpreting borderline results.

The biometric processing component 204 may utilize a parallel processing architecture that runs biometric analysis alongside neurocognitive assessment, to produce biometric adjusted neurocognitive assessment output 708. To do so, it may implement synchronized data streams with timestamp alignment at sub-second precision. It may employ a state machine architecture that coordinates assessment flow based on biometric feedback. It may utilize a secure communication protocol for exchanging sensitive health data between components. It further may implement graceful degradation to maintain core assessment functionality when biometric data is unavailable or incomplete.

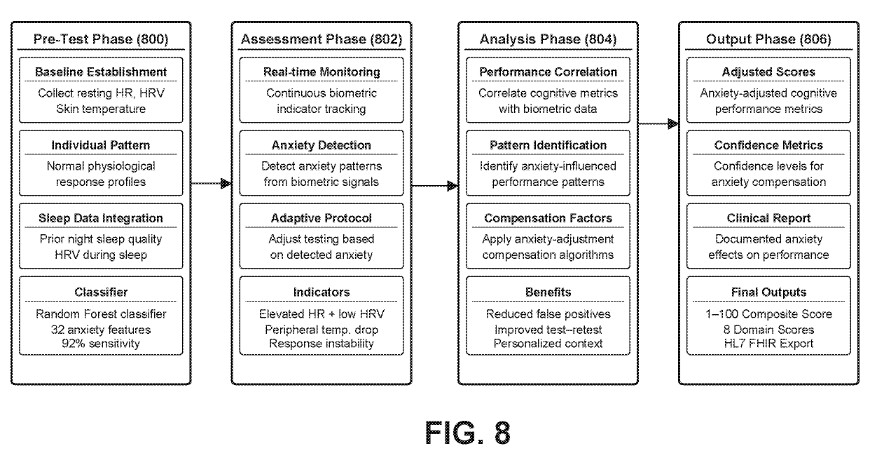

FIG. 8 shows an example of a process that may be performed by the anxiety detection engine 706a. The process is organized into four sequential phases: a pretest phase 800, an assessment phase 802, an analysis phase 804, and an output phase 806. The pre-test phase 800 establishes the foundation through baseline measurements of physiological indicators (heart rate, HRV, skin temperature) and individual pattern recognition, while also incorporating sleep quality data for contextual interpretation. During the assessment phase 802, the system performs real-time monitoring of biometric indicators alongside anxiety detection using proprietary algorithms, potentially adapting testing protocols when significant anxiety is detected. The analysis phase 804 correlates neurocognitive performance with physiological state, identifying anxiety-influenced patterns and applying appropriate compensation factors. Finally, the output phase 806 delivers anxiety-adjusted neurocognitive performance metrics with confidence ratings and clinical reports documenting how anxiety affected test performance. FIG. 8 also highlights key biometric indicators tracked by the system, specific anxiety pattern indicators (elevated heart rate, reduced HRV, peripheral temperature decrease, response irregularity), and benefits including reduced false positives, improved test-retest reliability, and personalized assessment context.

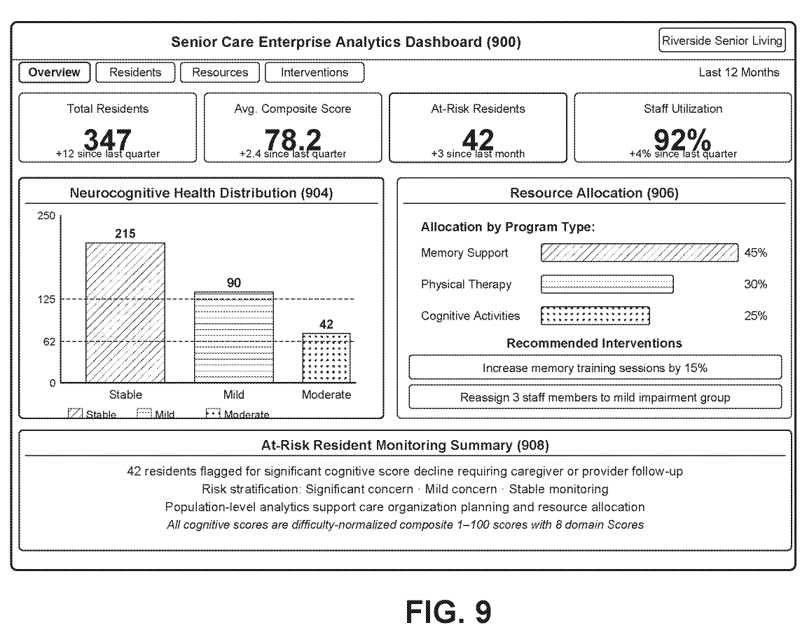

The AI engine 208 may generate an enterprise dashboard type user interface for senior care organizations. FIG. 9 illustrates an example of such an enterprise dashboard. The illustrated enterprise dashboard displays key metrics and graphics for a particular (fictional) senior living facility named Riverside Senior Living in this example. The metrics include a total resident count, average neurocognitive score, and number of at-risk residents with trend indicators showing changes since previous periods. The Neurocognitive Health Distribution chart 904 segments residents by neurocognitive status (215 stable, 90 mild impairment, 42 moderate impairment), while the Resource Allocation pie chart 906 breaks down current intervention types (Memory Support, Physical Therapy, Neurocognitive Activities). The dashboard provides data-driven recommendations for optimizing care, such as increasing memory training sessions by a particular percentage and reassigning a particular number of staff members to address needs in the mild impairment group. This enterprise dashboard thereby enables senior care facilities to identify residents needing intervention, allocate resources effectively, track neurocognitive health trends across their population, and make evidence-based staffing and programming decisions.

### VIII. Multi-Site Management User Interface

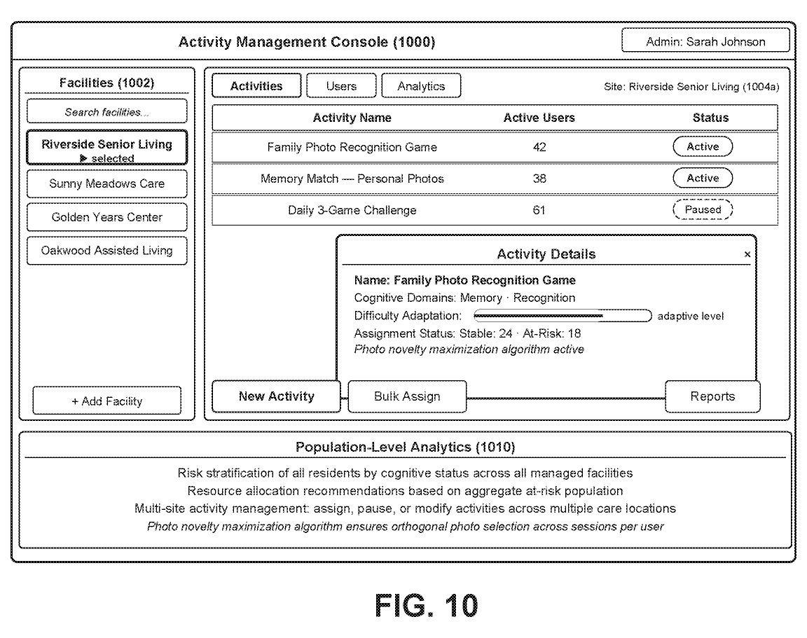

The AI engine 208 may also generate a multi-site management user interface that enables administrators to coordinate neurocognitive activities across multiple senior care facilities. FIG. 10 depicts an example of a display screen of such a multi-site management user interface neurocognitive. An administrator, Sarah Johnson, is shown managing the Activity Management Console, which provides a centralized control panel for deploying and monitoring the system’s neurocognitive activities across different locations. The left panel displays a list of facilities under management (in this example, Riverside Senior Living, Sunny Meadows Care, Golden Years Center, and Oakwood Assisted Living), while the main panel shows an Activity Details pop-up for a "Family Photo Quiz" neurocognitive assessment. That activity targets Memory and Recognition neurocognitive domains with customizable difficulty adaptation settings. The assignment status shows distribution across resident neurocognitive profiles (e.g., 24 stable residents, 18 at-risk residents). The display screen includes functionality for creating new activities, performing bulk assignments across facilities, and generating comprehensive reports. The management console streamlines the implementation of standardized neurocognitive health monitoring across enterprise organizations while allowing for customization based on facility-specific needs and resident populations.

### IX. Process Flows

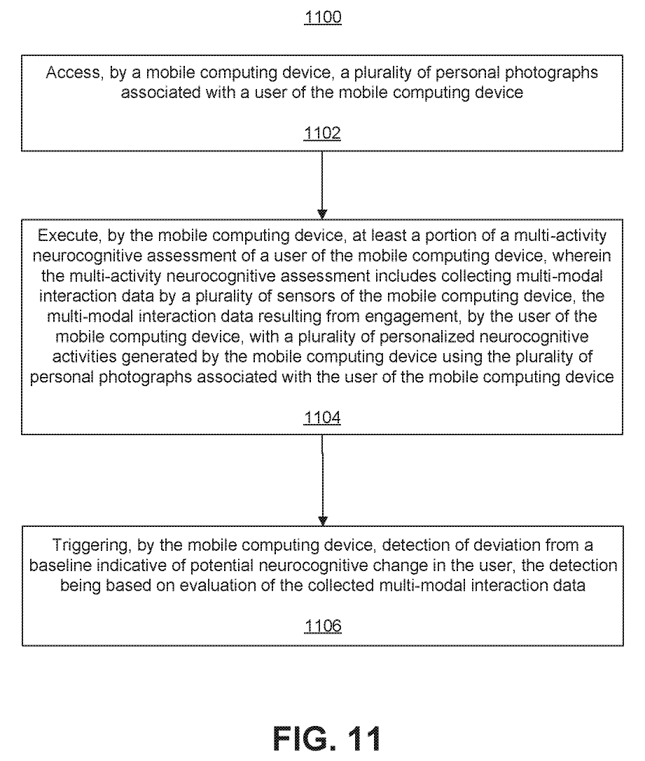

FIG. 11 is a flow diagram illustrating an example of a process 1100 that may be performed by a mobile computing device as part of a neurocognitive assessment of a user, according to some implementations. The process 1100 outlines the high-level steps taken by the mobile application to facilitate the collection of multi-modal interaction data using personalized content.

The process 1100 begins at step 1102, where the mobile computing device accesses a plurality of personal photographs associated with a user of the device. These photographs may be retrieved from the device's local camera roll or a cloud-based photo library and serve as the foundation for the assessment activities.

At step 1104, the mobile computing device executes at least a portion of a multi-activity neurocognitive assessment of the user. As part of this assessment, the mobile device collects multi-modal interaction data via a plurality of on-device sensors. This interaction data results from the user's engagement with a plurality of personalized neurocognitive activities (e.g., memory match or chronological games) that were generated by the device using the previously accessed personal photographs. The sensors of the mobile device involved in this collection may include a touchscreen and microphone, and optionally may further include an accelerometer and/or camera of the mobile device, thereby capturing a wide array of behavioral and physiological biomarkers.

Finally, at step 1106, the mobile computing device triggers detection of deviation from a baseline indicative of potential neurocognitive change in the user, the detection being based on evaluation of the collected multi-modal interaction data. In some implementations and/or instances, this triggering may involve invoking an evaluation process that also is performed in the mobile device. In other implementations and/or instances, the triggering involves causing transmission of the multimodal interaction data (or summary/digest data representative of the multimodal interaction data) to one or more remote server computers that perform the evaluation.

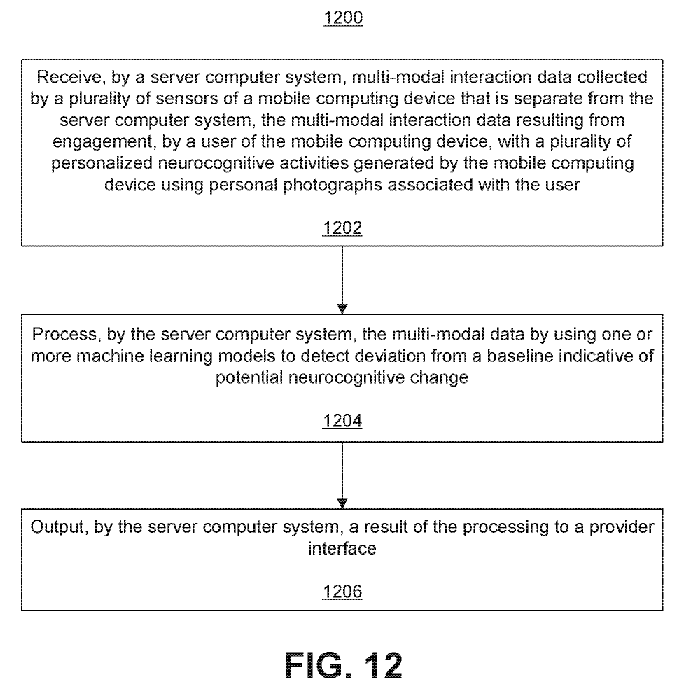

FIG. 12 is a flow diagram illustrating an example of a process 1200 that may be performed by one or more server computers as part of a neurocognitive assessment of a user, complementary to the process in FIG. 11. The server process 1200 is complementary to the mobile-device process 1100 in instances and/or implementations where the mobile device does not perform the entire neurocognitive assessment of the user.

The process 1200 begins at step 1202, where the server computer system receives multi-modal interaction data. This data has been collected by a plurality of sensors on a mobile computing device that is separate from the server system. As previously described, this interaction data results from the user’s engagement with a plurality of personalized neurocognitive activities that were generated by the mobile device using personal photographs associated with that specific user. The received data may include high-dimensional feature vectors representing temporal, spatial, behavioral, and speech-based biomarkers extracted during the assessment session.

At step 1204, the server computer system processes the multi-modal interaction data. This processing involves using one or more machine learning models to detect deviations from a longitudinal per-user baseline. For example, by comparing the current session data against the user’s own historical performance—rather than population norms—the system can identify subtle changes in neurocognitive function, such as shifts in learning curves, reaction time decay, or verbal fluency, which may be indicative of potential neurocognitive change or decline.

Finally, at step 1206, the server computer system outputs a result of the processing to a provider interface. This output may take the form of a normalized composite score, multiple domain-specific scores (memory, attention, executive function, etc.), longitudinal trend visualizations, or clinical alerts. The provider interface allows clinicians or caregivers to monitor the user’s neurocognitive health over time and receive timely notifications if significant deviations from the baseline are detected.

### X. Example Hardware Implementation

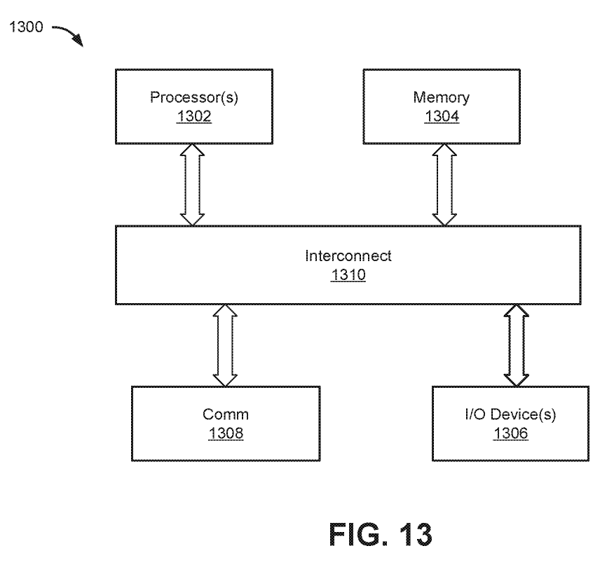

FIG. 13 is a block diagram of a computer system that may be used to implement some or all of the techniques introduced above, or any portion(s) thereof. For example, the computer system 1300 may represent the architecture of a mobile device, personal computer, server computer, or any other processing device mentioned herein. The computer system 1300 includes one or more processors 1302, one or more memories 1304, one or more input/output (I/O) devices 1306, and one or more communication interfaces 1308, all connected to each other through an interconnect 1310. The processor(s) 1302 control the overall operation of the computer system 1300, including controlling its constituent components. Each of the processor(s) 1302 may be or include one or more programmable microprocessors (e.g., one or more central processing units (CPUs), graphics processing units (GPUs), tensor processing units (TPUs), or the like), programmable logic devices (PLDs), field programmable gate arrays (FPGAs), application-specific integrated circuits (ASICs), etc.

The one or more memories 1304 store data and executable instructions (e.g., software and/or firmware), which may include software and/or firmware for performing the techniques introduced above. The one or more memories 1304 may include main memory and/or mass storage of the computer system 1300 and may be or include any of various forms of random access memory (RAM), read-only memory (ROM), volatile memory, nonvolatile memory, or any combination thereof. For example, the one or more memories 1304 may be or include dynamic RAM (DRAM), static RAM (SDRAM), flash memory, one or more disk-based hard drives, etc.

The I/O devices 1306 provide access to the computer system 1300 by human user, and may be or include, for example, a display monitor, audio speaker, keyboard, touch screen, mouse, microphone, trackball, etc. The communications interface 1308 enables the computer system 1300 to communicate with one or more external devices (e.g., one or more servers and/or clients of the computer system 1300) via a network connection and/or direct connection. The communications interface 1308 may be or include, for example, a Wi-Fi transceiver, Bluetooth transceiver, Ethernet adapter, Universal Serial Bus (USB) adapter, or the like. The interconnect 1310 may be or include, for example, one or more buses, bridges or adapters, such as a system bus, peripheral component interconnect (PCI) bus, PCI extended (PCI-X) bus, USB, or the like.

Unless contrary to physical possibility, it is envisioned that (i) the methods/steps described herein may be performed in any sequence and/or in any combination, and that (ii) the components of respective implementations may be combined in any manner.

Software or firmware to implement the techniques introduced here may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, tablet computer, personal digital assistant (PDA), manufacturing tool, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc. s

Unless contrary to physical possibility, it is envisioned that (i) the methods/steps described herein may be performed in any sequence and/or in any combination, and that (ii) the components of respective implementations may be combined in any manner.

The machine-implemented operations described above can be implemented by programmable circuitry programmed/configured by software and/or firmware, or entirely by special-purpose circuitry, or by a combination of such forms. Such special-purpose circuitry (if any) can be in the form of, for example, one or more application-specific integrated circuits (ASICs), programmable logic devices (PLDs), field-programmable gate arrays (FPGAs), system-on-a-chip systems (SOCs), etc.

Software or firmware to implement the techniques introduced here may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing tool, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc.

The term "logic", as used herein, means: a) special-purpose hardwired circuitry, such as one or more application-specific integrated circuits (ASICs), programmable logic devices (PLDs), field programmable gate arrays (FPGAs), or other similar device(s); b) programmable circuitry programmed with software and/or firmware, such as one or more programmed general-purpose microprocessors, digital signal processors (DSPs) and/or microcontrollers, system-on-a-chip systems (SOCs), or other similar device(s); or c) a combination of the forms mentioned in a) and b).

Any or all of the features and functions described above can be combined with each other, except to the extent it may be otherwise stated above or to the extent that any such implementations may be incompatible by virtue of their function or structure, as will be apparent to persons of ordinary skill in the art.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A method comprising:
accessing, by a mobile computing device, a plurality of personal photographs associated with a user of the mobile computing device;
executing, by the mobile computing device, at least a portion of a multi-activity neurocognitive assessment of a user of the mobile computing device, wherein the multi-activity neurocognitive assessment includes repeatedly collecting multi-modal interaction data by a plurality of sensors of the mobile computing device, the multi-modal interaction data resulting from engagement, by the user of the mobile computing device, with a plurality of personalized neurocognitive activities generated by the mobile computing device using the plurality of personal photographs associated with the user of the mobile computing device; and
triggering, by the mobile computing device, detection of deviation from a baseline indicative of potential neurocognitive change in the user, the detection being based on evaluation of the collected multi-modal interaction data.

2. The method of claim 1, wherein the multi-modal data comprises:
touchscreen input data acquired from a touchscreen of the mobile computing device; and
speech data acquired from a microphone of the mobile computing device.

3. The method of claim 2, wherein the multi-modal data further comprises eye motion data and/or facial expression data acquired by a camera of the mobile computing device.

4. The method of claim 3, wherein the multi-modal data further comprises data indicative of hand tremor frequency and amplitude acquired by an accelerometer of the mobile computing device.

5. The method of claim 2, wherein the multi-modal data further comprises data indicative of a movement disorder.

6. The method of claim 1, wherein the multi-activity neurocognitive assessment comprises repeatedly administering, by the mobile computing device, a multi-activity challenge involving a plurality of different activities, to the user.

7. The method of claim 6, wherein the plurality of different activities comprise:
a visual pattern matching activity;
a jigsaw puzzle activity; and
a memory story narration activity requiring a spoken response by the user.

8. A mobile computing device comprising:
a touchscreen;
a microphone;
a wireless transceiver configured to enable communications over a wireless communication network;
at least one processor; and
at least one memory storing instructions, execution of which by the at least one processor causes the mobile computing device to perform a process including
accessing a plurality of personal photographs associated with a user of the mobile computing device;
executing at least a portion of a multi-activity neurocognitive assessment of a user of the mobile computing device, wherein the multi-activity neurocognitive assessment includes repeatedly collecting multi-modal interaction data by a plurality of sensors of the mobile computing device, including the touchscreen in the microphone, the multi-modal interaction data resulting from engagement, by the user of the mobile computing device, with a plurality of personalized neurocognitive activities generated by the mobile computing device using the plurality of personal photographs associated with the user of the mobile computing device; and
triggering detection of deviation from a baseline indicative of potential neurocognitive change in the user, the detection being based on evaluation of the collected multi-modal interaction data.

9. The mobile computing device of claim 8, wherein the multi-modal data further comprises at least one of:
eye motion data and/or facial expression data acquired by a camera of the mobile computing device; or
data indicative of hand tremor frequency and amplitude acquired by an accelerometer of the mobile computing device.

10. The mobile computing device of claim 8, wherein the multi-activity neurocognitive assessment comprises repeatedly administering a multi-activity challenge protocol involving a plurality of different activities, to the user.

11. The mobile computing device of claim 10, wherein the multi-activity challenge protocol comprises:
a visual pattern matching activity;
a jigsaw puzzle activity; and
a memory story narration activity requiring a spoken response by the user.

12. A method comprising:
receiving, by a server computer system, multi-modal interaction data collected by a plurality of sensors of a mobile computing device that is separate from the server computer system, the multi-modal interaction data resulting from engagement, by a user of the mobile computing device, with a plurality of personalized neurocognitive activities generated by the mobile computing device using personal photographs associated with the user;
processing, by the server computer system, the multi-modal data by using one or more machine learning models to detect deviation from a baseline indicative of potential neurocognitive change; and
outputting, by the server computer system, a result of the processing to a provider interface.

13. The method system of claim 12, wherein the provider interface comprises a healthcare provider interface configured to enable controlled access to a result of the processing by a healthcare provider.

14. The method of claim 12, wherein the result includes a difficulty-normalized composite neurocognitive health score and a plurality of domain scores related to the user of the mobile computing device, the plurality of domain scores corresponding to a plurality of neurocognitive domains.

15. The method of claim 12, wherein the processing comprises evaluating temporal response patterns and generating accuracy metrics related to the engagement by the user with the personalized neurocognitive activities generated by the mobile computing device.

16. The method of claim 12, such that the personalized neurocognitive activities generated by the mobile computing device include a multi-activity challenge protocol.

17. The method of claim 16, such that the multi-activity challenge protocol comprises:
a visual pattern matching activity;
a jigsaw puzzle activity; and
a memory story narration activity requiring a spoken response by the user.

18. The method of claim 12, further comprising:
qualifying, by the server system, each photograph of the plurality of personal photographs for use in one or more of the plurality of personalized neurocognitive activities, by estimating a depth of subject matter depicted in the photograph.

19. The method of claim 12, further comprising:
maintaining, by the server system, a longitudinal per-user performance baseline for the user; and
generating, by the server system, the result of the processing by evaluating the performance of the user in the personalized neurocognitive activities against the per-user performance baseline for the user.

20. The method of claim 12, wherein the multi-modal data comprises:
touchscreen input data acquired from a touchscreen of the mobile computing device; and
speech data acquired from a microphone of the mobile computing device.

21. The method of claim 20, wherein the multi-modal data further comprises eye motion data and/or facial expression data acquired by a camera of the mobile computing device.

22. The method of claim 21, wherein the multi-modal data further comprises data indicative of hand tremor frequency and amplitude acquired by an accelerometer of the mobile computing device.

23. The method of claim 20, wherein the multi-modal data further comprises data indicative of a movement disorder.

24. A server system comprising;
at least one processor;
at least one machine-readable program medium storing instructions, execution of which by at least one processor causes the server system to perform a process including
receiving multi-modal interaction data collected by a plurality of sensors of a mobile computing device that is separate from the server system, the multi-modal interaction data resulting from engagement, by a user of the mobile computing device, with a plurality of personalized neurocognitive activities generated by the mobile computing device using personal photographs associated with the user;
processing the multi-modal data by using one or more machine learning models to detect deviation from a baseline indicative of potential neurocognitive change; and
outputting a result of the processing to a user interface.

25. The server system of claim 24, wherein the result includes a difficulty-normalized composite neurocognitive health score and a plurality of domain scores related to the user of the mobile computing device, the plurality of domain scores corresponding to a plurality of neurocognitive domains.

26. The server system of claim 24, wherein the processing comprises evaluating temporal response patterns and generating accuracy metrics related to the engagement by the user with the personalized neurocognitive activities generated by the mobile computing device.

27. The server system of claim 24, such that the personalized neurocognitive activities generated by the mobile computing device include repeating a multi-activity challenge protocol, including:
a visual pattern matching activity;
a jigsaw puzzle activity; and
a memory story narration activity requiring a spoken response by the user.

28. The server system of claim 24, further comprising:
qualifying each photograph of the plurality of personal photographs for use in one or more of the plurality of personalized neurocognitive activities, by estimating a depth of subject matter depicted in the photograph.

29. The server system of claim 24, wherein the multi-modal data comprises:
touchscreen input data acquired from a touchscreen of the mobile computing device; and
speech data acquired from a microphone of the mobile computing device.

30. The server system of claim 29, wherein the multi-modal data further comprises at least one of:
eye motion data and/or facial expression data acquired by a camera of the mobile computing device; or
data indicative of hand tremor frequency and amplitude acquired by an accelerometer of the mobile computing device.
